(19) United States
(12) Patent Application Publication
(10) Pub. No.: US 2009/0306454 A1
(43) Pub. Date: Dec. 10, 2009

(54) Devices and Methods for Stimulation of Tissue

(75) Inventors: Kimberly P. Cockerham, Los Altos, CA (US); Harvey A. Fishman, Menlo Park, CA (US); Anthony Liu, San Mateo, CA (US); Alissa M. Fitzgeral, San Francisco, CA (US); Dorian Liepmann, Lafayette, CA (US); Benjamin W. Chul, Mountain View, CA (US); Michael F. Marmor, Stanford, CA (US); Wentai Liu, Santa Cruz, CA (US); Juan G. Santiago, Stanford, CA (US)

Correspondence Address: LUMEN PATENT FIRM, 350 Cambridge, Suite 100, PALO ALTO, CA, 94306, US

(73) Assignee: Stanford University, Palo Alto, CA (US)

(21) Appl. No.: 12/083,586

(22) PCT Filed: May 3, 2006
(86) PCT No.: PCT/US06/16812

§ 371 Date: Jul. 7, 2009

Related U.S. Application Data
(60) Provisional application No. 60/734,859, filed on Nov. 8, 2005.
(60) Provisional application No. 60/788,557, filed on Mar. 30, 2006.

(51) Int. Cl. A61N 1/36; A61N 2/00

(52) U.S. Cl. 600/9; 604/20

(57) ABSTRACT

Devices, systems and methods are provided for directly stimulating tissues, particularly muscle tissues, to modulate muscle contractions (i.e. provide reanimation of the muscle or to suppress undesired muscle contractions). Reanimation of muscles may be desired when damage to the brain, nervous system or neuromuscular junctions have occurred, causing a muscle tissue to lack sufficient motor control. Suppression of muscle contractions may be desired in situations of pathologically hyperactive muscles, such as in conditions of muscle spasm (e.g. blepharospasm and hemifacial spasm) or muscle dystonia. Direct stimulation is achieved by delivering a chemical agent directly to the muscle tissue, particularly the motor end plate, bypassing the nerves and neuromuscular junctions which may be damaged or diseased. Implanted hybrid chemical and electromagnetic stimulation devices can modulate muscle contraction in response to signals from a controller.

### CROSS-REFERENCES TO RELATED APPLICATIONS

NOT APPLICABLE

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

Movement in the human body is governed by the nervous system, and is expressed in the activity of the muscular system. The desire to initiate movement is formed in the brain, and signals are sent from sets of nerves in the brain to the appropriate muscles in a complex coordinated fashion in order to produce the desired movement. The nerves in the brain typically send signals to these muscles via one or several “connector” nerves which form a pathway from the brain to the muscles of interest. All nerves and muscles have “receiver’ sites for receiving such signals. All nerves also have a “signal sending” end for communicating such signals to other nerves, or to organs at the end of the pathway such as muscles.

Nerves can vary greatly in length from microscopic distances to the length of the entire leg of a person. Once the receiver end of a nerve is activated by a neurotransmitter, the signal is communicated along the distance of a nerve by production of an electric signal. The electric signal starts at the receiver end, and travels the length of the nerve to the signal sending end. Once the electric signal reaches the signal sending end of the nerve, a series of events leads to the release of neurotransmitter to the next nerve, or to the organ at the end of the pathway. Thus electric signals also play a key role in communicating signals from one part of the body to another.

Nerve cells which innervate skeletal-muscle fibers are known as motor neurons and their cell bodies are located in either the brainstem or the spinal cord. FIG. 1 illustrates a motor neuron 10 comprising a cell body 12 and at least one axon 14 extending therefrom. The axons 14 of motor neurons 10 are myelinated (i.e. encased by a myelin sheath 16 which assists in propagating action potentials) and are the largest-diameter axons in the body. They are therefore able to propagate action potentials at high velocities, allowing signals from the central nervous system to be transmitted to skeletal-muscle fibers with minimal delay.

The myelin sheath 16 surrounding the axon 14 of a motor neuron 12 ends near the surface of a muscle fiber 18, as illustrated in FIG. 2. The axon 14 divides into a number of short processes that lie embedded in grooves on the surface of the muscle fiber 18. The terminal portion of the axon 14 is called the axon terminal 20. The region of the muscle-fiber 18 that lies directly under the axon terminal 20 has special properties and is known as the motor end plate 22. The junction of the axon terminal 20 with the motor end plate 22 is known as a neuromuscular junction 24. The neuromuscular junction 24 is schematically illustrated in FIG. 1. Typically, a small space exists between the axon terminal 20 and the motor end plate 22; the space is termed a synaptic cleft 26. Together, the axon terminal 20, neuromuscular junction 24 and motor end plate 22 form a synapse. Nerve impulses are transmitted to the muscle fiber 18 via chemical communication with such synapses.

FIG. 3 provides a schematic illustration of a synapse. The axon terminal 20 of the motor neuron 10 contains membrane-bound vesicles 30. The vesicles 30 contain the chemical transmitter acetylcholine 32. When an action potential on a motor neuron 10 arrives at the axon terminal 20, it depolarizes the nerve plasma membrane, opening voltage-sensitive calcium channels, and thus allowing calcium ions to diffuse into the axon terminal 20. This calcium triggers the release of, by exocytosis, of acetylcholine 32 from the vesicles 30, as shown, into the extracellular cleft 26 separating the axon terminal 20 and the motor end plate 22. The acetylcholine 32 diffuses across the cleft 26 and binds to receptor sites 34 on the motor end plate 22. The binding of acetylcholine 32 activates the receptor 34 which opens ion channels in the end-plate membrane. This induces a local depolarization of the motor end plate 22 which initiates an action potential which propagates over the surface of the muscle fiber 18.

Referring back to FIG. 2, when a skeletal muscle fiber 18 is activated cross bridges (not shown) in the thick filaments 40 bind to actin in the thin filaments 42 and undergo a conformational change that propels the thin filaments 42 toward the center of a sarcomere 44. Chemical activation of the cross bridges within muscles is termed contraction. Contraction is the basis of volutional and reflexive muscle motion. Following contraction, the initiation mechanisms are turned off, and tension generation declines, producing relaxation of the muscle fiber.

Referring again to FIG. 3, in addition to receptor sites 34 for acetylcholine 32, the motor end plate 22 on a muscle fiber 18 also contains the enzyme acetylcholinesterase at its surface. This enzyme breaks down acetylcholine 32. Acetylcholine 32 bound to receptor sites 34 is in equilibrium with free acetylcholine in the cleft 26 between the nerve and muscle membranes. As the concentration of free acetylcholine 32 falls because of its breakdown by acetylcholinesterase, less acetylcholine 32 will bind to the receptor sites 34. When the receptor sites 34 no longer contain bound acetylcholine 32, the ion channels in the end plate 22 close and the depolarized end plate 22 returns to its resting potential and can respond to the arrival of a new burst of acetylcholine 32 released by another nerve action potential.

It may be appreciated that within a muscle, the axon of a motor neuron divides into many branches, each branch forming a single junction with a muscle fiber. Thus, a single motor neuron innervates many muscle fibers, but each muscle fiber has only one nerve junction and therefore is controlled by only one motor neuron. A motor neuron plus the muscle fibers it innervates is called a motor unit. Although the muscle fibers in a single motor unit are all located in one muscle, they are scattered throughout the muscle and therefore are not lying adjacent to each other. When an action potential is produced in a single motor neuron, all of the muscle fibers in its motor unit contract.

Nerve damage or dysfunction at any point along the nervous system (e.g. brainstem, peripheral nerve, neuromuscular junction) can disrupt the signal transmission pathways and leave muscles unable to contract normally. Such damage can occur due to a variety of factors, such as demyelination (destruction of the myelin sheath), conduction block (the impulse is blocked somewhere along the nerve pathway), and axonopathy (damage to the nerve axon). Some associated diseases and conditions include alcoholic neuropathy, diabetic neuropathy, nerve effects of uremia (from kidney failure), traumatic injury to a nerve, Guillain-Barre syndrome, diphtheria, carpal tunnel syndrome, brachial plexopathy, Charcot-Marie-Tooth disease (hereditary), chronic inflammatory polyneuropathy, common peroneal nerve dysfunction, distal median nerve dysfunction, femoral nerve dysfunction, myasthenia gravis, Paraneoplastic syndromes, Friedreich's ataxia, general paresis, Lambert-Eaton Syndrome, Amyotrophic Lateral Sclerosis (“ALS”), mononeuritis multiplex, primary amyloid, radial nerve dysfunction, sciatic nerve dysfunction, secondary systemic amyloid, sensorimotor polyneuropathy, tibial nerve dysfunction, ulnar nerve dysfunction, to name a few.

Muscles that have lost their input from the nervous system due to nerve damage are unable to contract normally and eventually become atropic. Researchers have attempted to artificially stimulate contraction in muscles using electric stimulation. Electric current of the proper parameters applied directly to nerves or muscles causes the nerves or muscles to depolarize (become activated). This production of electric signal by artificial means leads to activation of the nerve pathway ending in muscle contraction, or directly causes the muscle itself to contract.

Three muscles are involved in the production of synchronous eyelid opening and closing: the levator, Mueller's and orbicularis oculi muscles. The levator and Mueller's muscle are innervated by the third cranial nerve and sympathetic, respectively, and work in concert to open the eyelid. In contrast, the orbicularis oculi muscle is innervated by the seventh nerve and is used in eyelid closure. When the eyelid is closed, the orbicularis oculi is stimulated and the levator/Mueller's muscles are inhibited. Failure to inhibit the antagonist muscles can prevent eyelid closure. Simultaneous stimulation of the agonist and antagonist may result in spastic twitching without eyelid closure.

The nerve to muscle ratio in the orbicularis oculi may be the most abundant in the body (approximately 1:3). Also, the tissues are extremely well vascularized allowing abundant oxygenation and effective toxin removal. In addition, the motor units have an unusual “grape-like” morphology. Further, the facial muscles and extraocular muscles may have the shortest contraction time in the body (7 msec) and the highest potential frequency of contraction (number of events per second). The resting tone of the orbicularis oculi, for example, may have a contraction frequency of about 50 contractions per second, which may rise to 170 contractions per second or more. This high frequency of contraction combined with the low voltage system of the orbicularis oculi muscle can allow rapid, fine and sustained orbicularis oculi movements that may be unique in the body. The depolarization seen clinically on an EMG can precede the simultaneous blinking of both eyelids by only about 20 microseconds.

Electrical stimulation of peripheral muscles has been utilized to inhibit muscle atrophy in patients with temporary nerve dysfunction or following nerve grafting procedures. However, reanimation of muscle units are not commonly used, at least in part because of shortcomings of various neural tissue interfaces. Practical limitations are many. For example, transcutaneous electrodes are typically passed through the skin to stimulate the underlying muscles. These may be awkward to affix and can produce unpleasant cutaneous sensations due to high currents. Percutaneously inserted wire electrodes may be cosmetically unappealing, prone to breakage and may be a potential conduit for infection. Fully implanted systems are often expensive and invasive to implant due to the need for lengthy leads. Moreover, electrode materials can degrade over time or become deactivated by scar tissue forming over them. Further, chronic electric stimulation can also desensitize the muscle or nerve tissue reducing the ability to stimulate at safe levels of electric current.

Several groups have also carried out clinical experiments to determine if stimulating retinal cells, the optic nerve bundle or cells of the visual cortex with microelectrode arrays can cause sensations of light in individuals impaired with age-related macular degeneration. The electrical fields produced by the microelectrode arrays stimulate relatively large regions containing numerous neuronal and glial cells. Although these trials have demonstrated that vision is recoverable in a limited fashion, major challenges remain. Due to the size and difficulties in placement of most available electrodes, imprecise electric field stimulation extending of long distances is used to depolarize neurons. Such methods often require excessive stimulation, which may be harmful, leading to inflammation of the stimulated region and even to excessive growth of glial cells or gliosis.

Consequently, other methodologies to provide perception of light in the retina have been developed that do not rely on electrical stimulation. Such methodologies included the creation of an artificial synapse to replace damaged or dysfunctional synapses. Such artificial synapses may be used when neurons are still viable and active yet lack connections to other neurons for receiving signals. By artificially stimulating such viable neurons, there is believed to be an opportunity to provide responses to visual signals so that the brain can interpret the signals and provide a visual output of the signals, giving the experience of seeing.

As mentioned, such artificial synapses rely on the presence of viable neurons among other elements of the visual anatomy such as the brain and visual pathways. However, in many conditions of disease or injury, these elements, particularly neurons, are not viable and are unable to transmit signals when stimulated. Therefore, it is desired to provide devices, systems and methods which activate all types of tissue, including denervated tissues. In particular, it is desired to provide modulation of muscles which lack appropriate pathways for natural stimulation and control. Further, it is desired to provide modulation of facial muscles, and in particular, of the eyelids. Desirably functioning eyelids are critical to the health, appearance and well being of a patient yet provide unique challenges. At least some of these objectives will be provided by the present invention.

### BRIEF SUMMARY OF THE INVENTION

Devices, systems and methods are provided for directly stimulating tissues, particularly muscle tissues, to modulate muscle contractions (i.e. provide reanimation of the muscle or to suppress undesired muscle contractions). Exemplary embodiments provide implanted hybrid chemical and electromagnetic stimulation devices. Reanimation of muscles may be desired when damage to the brain, nervous system or neuromuscular junctions have occurred, causing a muscle tissue to lack sufficient motor control. Suppression of muscle contractions may be desired in situations of pathologically hyperactive muscles, such as in conditions of muscle spasm (e.g. blepharospasm and hemifacial spasm) or muscle dystonia. Stimulation may also be used to treat hypotonic muscles. Direct stimulation may be achieved at least in part by delivering a chemical agent directly to the muscle tissue, particularly the motor end plate, bypassing the nerves and neuromuscular junctions which may be damaged or diseased. Direct stimulation leads to muscle contraction or relief of existing muscle contraction, providing movement of a body part, resting muscle tone, muscle relaxation or other desired effects. Moreover, chemical stimulation may be used as the threshold for stimulation either by electrical or chemical means, with many embodiments employing hybrid chemical and electromagnetic stimulation, optionally in the form of electrochemical stimulation, to modulate contraction of a muscle. This improves function of the tissue and allows the patient to at least partially regain native movement and/or appearance in the affected area, relieving a variety of symptoms, suspending the progression of disease and disability, and improving quality of life.

The chemical agent is delivered by a delivery device that releases, such as ejects, a reproducible small volume of the chemical agent, typically directly to the dysfunctional muscle. The device typically contains a plurality of reservoirs containing one or more chemical agents, such as chemical transmitters, neurotransmitters, elements (such as calcium), trophic factors and other pharmaceutic substances. In preferred embodiments, the chemical agent comprises acetylcholine, a chemical transmitter. Acetylcholine binds to receptor sites on motor end plates, inducing local depolarization of the motor end plate. This initiates an action potential which propagates over the surface of the associated muscle fiber leading to muscle contraction. Analogs of acetylcholine may also be used, particularly chorbechol (which has a sufficient stable life at body temperature to facilitate use from an implanted reservoir after 15 days or more, or 30 days or more). In other embodiments, the chemical agent comprises acetylcholinesterase, an enzyme which breaks down acetylcholine leading to depolarization of the end plate and return to its resting potential. Alternatively, the chemical agent may comprise an element, such as calcium. Such a chemical agent may be suitable for patients having intact neuromuscular junctions yet deficiencies in other aspects of the neural system. Calcium may be released or ejected onto the muscle fibers which trigger release, via exocytosis, of acetylcholine. The released acetylcholine binds to receptor sites on motor end plates, inducing local depolarization of the motor end plate which initiates an action potential that propagates over the surface of the associated muscle fiber leading to muscle contraction. Likewise, the reservoirs may contain chemical agents comprising growth factors or immunomodulators to prevent muscle atrophy and minimize any possibility of immune response to the implant. Neurotrophic chemicals can be applied to regenerate damaged tissue as well. Thus, the chemical agent is delivered directly to the motor end plate, bypassing the neuromuscular junction and/or the neuron.

In preferred embodiments, the chemical agent is drawn from each reservoir through microfluidic channels and ejected through orifices to the surrounding tissue. For example, the chemical agent may be moved by mechanical pressure which is created by, for example, a valve membrane, piston or expansion of a gas bubble created by electrolysis of water/hydrolysis or other chemical reaction, or by a pressurized chamber with valves to control outflow. In other embodiments, the chemical agent is moved through the microfluidic channels by electroosmosis or electrophoresis. Such delivery is achieved using electric fields without moving parts and can be used to efficiently control an array of stimulation sites. In other embodiments, this is achieved by electrolysis/hydrolysis piston systems. Delivery devices may optionally combine these and/or other systems. Chemical agent delivery will preferably be combined in a hybrid stimulation device with electromagnetic stimulation of the muscle tissue, such as by applying an electrical potential using an electrode, a magnetic stimulation using a coil or microcoil, or a combination of both

In one aspect of the present invention, a delivery device is provided for chemical stimulation of a muscle having a motor end plate. In one embodiment, the delivery device comprises a structure having at least one reservoir for holding at least one chemical agent. In this embodiment, the structure is adapted for positioning near the muscle and the structure is configured to deliver the at least one chemical agent directly to the motor end plate so as to cause an electrical change in the muscle. The electrical change may comprise initiation of an action potential or return of the muscle to its resting potential, to name a few. When the muscle controls movement of one or more eyelids, the electrical change in the muscle may cause movement of the one or more eyelids. An example of a muscle which moves one or more eyelids is the orbicularis oculi muscle. Thus, the structure may be configured for implantation within the one or more eyelids. It may be appreciated that when the muscle comprises a facial muscle, such as any muscles innervated by the facial nerve or other nerves, the electrical change in the muscle may cause contraction or relaxation of the facial muscle.

In some embodiments, the structure of the delivery device includes at least one microfluidic channel extending from each reservoir to an associated orifice through which the at least one chemical agent is delivered to the muscle. The delivery device may further include at least one electrode configured to assist in transport of the at least one chemical agent through the at least one microfluidic channel. Alternatively or in addition, the delivery device may further comprise at least one pump configured to assist in transport of the at least one chemical agent through the at least one microfluidic channel. In some embodiments, the delivery device further comprises access lines extending between a main reservoir and the at least one reservoir. Exemplary embodiments will combine chemical agent delivery with electromagnetic stimulation of the tissue, optionally including a stimulation electrode in a hybrid electromagnetic/chemical stimulation device.

In another aspect of the present invention, systems are provided for chemical stimulation of a muscle. In some embodiments, such systems include a delivery device comprising a structure having at least one reservoir for holding at least one chemical agent, wherein the structure is adapted for positioning near the muscle and for releasing the at least one chemical agent toward the muscle, and a controlling device which provides control signals to the delivery device, wherein the control signals control the release of the at least one chemical agent. The controlling device may comprise a microprocessor and memory, wherein the memory includes a program which drives the microprocessor. In such instances, the program may determine a pattern of release of the at least one chemical agent.

Typically the delivery device is adapted for implantation within the body during use. Many embodiments will comprise implanted hybrid electromagnetic and chemical stimulation devices. In some embodiments, the delivery device is adapted for implantation within one or more eyelids. The controlling device is often adapted for residing outside of the body during use.

In some embodiments, the system further comprises a sensing device adapted for positioning near another muscle, wherein the sensing device senses changes in the other muscle and provides feedback signals to the controlling device and wherein the control signals depend on the feedback signals. The sensing device may sense changes in voltage or movement of the another muscle, for example. In some embodiments, the another muscle comprises a contralateral muscle and wherein the control signals cause delivery of the at least one chemical agent to the muscle so as to synchronize the muscle with the contralateral muscle. Concurrent bilateral movements and the signals transmitted for initiating such movements (including signals transmitted to or from a single subnucleus, for example, to cause coordinated bilateral blinking of both eyes) may be advantageous for triggering muscle stimulation so as to avoid inhibition of the desired movement by an antagonist muscle. For example, the body's blink command signal may induce contraction of the orbicularis to close a properly functioning eye, and may also relax the levator muscles that keep both eyes open. That natural signal may not result in closure of the eye having a denervated muscle. Nonetheless, rather than attempting to blink the eyes at different times, it may be beneficial to blink the eye with the denervated muscle when the functional eye blinks to avoid inhibition of the blink by the antagonist levator muscles.

It may be appreciated that the systems may further comprise an additional delivery device comprising a structure having at least one reservoir for holding at least one chemical agent, wherein the structure is adapted for positioning near a second muscle and for releasing the at least one chemical agent toward the additional muscle, wherein the controlling device which provides additional control signals to the additional delivery device, wherein the additional control signals control the release of the at least one chemical agent from the additional delivery device.

In yet another aspect of the present invention, methods are provided for stimulating a muscle within a body. In some embodiments, the method includes implanting a delivery device within the body, the delivery device comprising a structure from which at least one chemical agent is releasable and wherein implanting comprises positioning the structure near the muscle, and activating the delivery device causing release of the at least one chemical agent to the muscle. Activating may comprise sending control signals to the delivery device. In some embodiments, sending control signals comprises positioning a controlling device within range of the delivery device so that the delivery device receives the control signals. It may be appreciated that activating may include causing the agent to release by degradation of a membrane or material, by gravitational pull or by any other mechanical, chemical or material means.

In some embodiments, the muscle controls movement of one or more eyelids. An example of a muscle which moves one or more eyelids is the orbicularis oculi muscle. Thus, the structure may be configured for implantation within the one or more eyelids. It may be appreciated that when the muscle may comprise any facial muscle, such as any muscles innervated by the facial nerve or other nerves, or any muscle or tissue.

In some embodiments, the desired motion, for example closure of the eyelid, may be achieved either through the direct chemical stimulation of an agonist, such as the muscle which closes the eyelid. The agonist in this example would be the orbicularis oculi. Alternatively, closure of the eyelid could be achieved through the inhibition of an antagonist(s), such as the muscle(s) which open the eyelid, the levator palpebrae and Mueller's muscle. Alternatively, closure of the eyelid (or some other movement) could be achieved through inhibition of the antagonist muscle(s) combined with simultaneous electric, chemical, or electrochemical stimulation of the agonist muscle. Inhibition of muscle activity could be achieved through the release of various chemical agents, including but not limited to acetylcholinesterase, or botulinum toxin.

In some embodiments, the methods further include positioning a sensing device so that the sensing device senses a change in another muscle within the body and provides feedback signals which assist in controlling activation of the delivery device. The other muscle may comprise a contralateral muscle.

It may be appreciated that the methods may further comprise implanting an additional delivery device within the body, the additional delivery device comprising a structure from which at least one chemical agent is releasable and wherein implanting the additional delivery device comprises positioning its structure near an additional muscle, and activating the additional delivery device causing release of the at least one chemical agent from the additional delivery device to the additional muscle. For example, the muscle may control movement of one or more eyelids and the additional muscle may comprises a muscle within a cheek.

It may also be appreciated that the delivery devices may provide a combination of chemical stimulation with more traditional or newly developed devices for electric stimulation of muscle in order to produce a desired modulation in muscle activity. Electric stimulation is well known to be able to stimulate muscle contraction, and is used in numerous medical and non-medical devices. Specialized electrical stimulation devices and methods described herein or otherwise configured for implantation in human and other patients may allow sustained muscle contraction modulation. The addition of chemical stimulation provides for a mechanism of stimulation which mimics the body's natural means of stimulating muscle, specifically through the use of neurotransmitters. Depending on the bioactive substance placed in the device for delivery to the target muscle(s), the device may also accomplish inhibition of unwanted muscle activity, such as in muscle spasm or dystonia. Hence, the devices, systems, and methods described herein will often include muscle stimulation electrodes (along with chemical stimulation structures) so as to allow electrochemical stimulation of muscle tissues, typically in response to a signal from a system control device.

In another aspect, the present invention provides a muscle stimulation system for modulating contraction in a muscle of a patient. The muscle stimulation system comprises a structure having a fluid transmission surface. A reservoir is provided for a chemical agent, and the reservoir is coupled with the surface so as to release fluid therethrough. A stimulation electrode is disposed along or near the surface. A controller is coupled with the stimulation electrode, the controller configured to transmit muscle stimulation signals from the stimulation electrode to the muscle when the structure is implanted in the patient with the surface adjacent to the muscle.

The fluid transmission surfaces of exemplary embodiments have a plurality of orifices disposed on an array of protrusions. The exemplary surfaces have an array of protruding microfluidic needles with sufficient length to extend through a fibrotic capsule. Encapsulation of the implant may be included in the tissue response to implantation, and such encapsulation could otherwise degrade system performance. Exemplary protrusions have lengths of over 100 μm. Optionally, a tissue-growth inhibiting agent can be released along the surface so as to inhibit encapsulation, avoid tissue ingrowth into the implant, limit implant-induced hyperplasia, and the like. Some embodiments may, for example, include drug eluting coatings similar to those of (or modified from those developed for) drug eluting stents. These coatings may be disposed along the fluid delivery surface, with exemplary coatings often comprising a matrix impregnated with the desired drug. Suitable drugs to limit detrimental tissue growth may include anti-inflammatory agents, anti-proliferative agents, chemotherapy drugs, anti-metabolites such as Fluorouracil (5FU), insulin-like growth factor (IGF-1), Mitomycin, and the like. Embodiments may apply an electrical potential to the fluid transmission surface to inhibit tissue growth. In some embodiments, the fluid transmission surface may border a permeable material or membrane.

The reservoir will often be coupled to the surface and/or orifices by a microfluidic channel system to effect controlled delivery of the chemical agent. The channel system can (in response to signals from the controller) deliver the agent to the at least one orifice with sufficient pressure to inhibit ingrowth of tissue into the orifice. For example, fluid may be directed to the orifice(s) with about 2 psi or more. The channel system will often have a pump to move the agent through the surface. Alternate systems may employ a pressurized fluid container coupled to a microvalve such as a solenoid valve or the like.

Any of a variety of muscle stimulation agents may be used, including those described above, with many embodiments employing acetylcholine or a nicotinic mimetic. For any systems that will contain a sufficient implanted quantity of a muscle stimulation chemical agent for use over more than two weeks (particularly more than a month), the chemical agent will often comprise a muscle stimulating analogue of acetylcholine having a stable life at body temperature longer than that of acetylcholine, such as carbachol. About 5 cc or less of carbachol (and/or other agent(s)) will generally be contained within the exemplary implanted system, and each muscle stimulation cycle may involve a release of a sufficiently small quantity that the carbachol effectively diffuses between cycles. Typically, less than about 200 μl of agent will be released for each stimulation cycle, often being from about 2 nl to about 100 μl, preferably being less than about 10 μl (for example, being from about 2 nl to about 10 μl).

The controller may induce corresponding release of the agent and electrical stimulation, often with the chemical stimulation being at least in part concurrent with the electrical stimulation, such as where the agent is ejected from the surface at the same time the electrode is energized. In some embodiments, the chemical agent may be directed to a muscle prior to energizing the electrode to pre-condition the muscle for electrical stimulation. Regardless, the chemical stimulation may significantly reduce the electrical potential for inducing muscular contraction (as compared to electrical stimulation alone), the electrochemical stimulation systems herein generally applying electrical stimulation of about 1.0 V or less to the muscle, often applying 0.5 V or less, typically with a pulse width of 200 ms or less for each contraction cycle. Such modest stimulation signals represent a significant decrease in (or even elimination of) patient pain that can otherwise result from electrical stimulation alone. The stimulation electrode(s) will often extend along the surface with a length sufficient for engaging a substantial portion of a corresponding overall dimension of the muscle to be engaged by that electrode. For example, the electrode may have a length of at least 20% of a length of the muscle, typically being at least 50%, preferably being 75% or more. Exemplary implantable structures may comprise thin structures having opposed major surfaces, with at least one (or both) of the major surfaces being the fluid transmission surface. Stimulation electrodes can be disposed along both of these major surfaces.

In addition to (or instead of) muscle contraction stimulation, embodiments may inhibit or counteract muscle atrophy, particularly after denervation. The chemical agent may, for example, comprise a trophic factor such as a growth factor, a growth-limiting factor antibody, or a combination thereof, and the implanted structure may release a sufficient quantity of the chemical agent(s) to effectively counteract or inhibit atrophy of the muscle. Exemplary trophic factors may comprise, for example, insulin-like growth factor (IGF-1), myostatin antibodies, or the like. At least some of the components of the system (including the agent delivery surface, orifices, some or all of the channel network, the reservoir, the controller, and/or the like) may be used to deliver both trophic factors and muscle stimulation agents. For example, before or after implantation, a trophic factor may be introduced into the reservoir, with chemical agent release initially counteracting atrophy of a denervated muscle. Thereafter, a muscle stimulation agent may be introduced into the same reservoir for subsequent electrochemical muscle stimulation. More complex multi-reservoir and/or multi-agent systems may also be provided.

The controller may initiate muscle contraction signals in response to contraction of a corresponding bilateral muscle, such as by including a muscle contraction sensor. The muscle contraction sensor may comprise an implantable microfluidic pH sensor implant generating electrical signals, a micro-electromechanical system (MEMS) acceleration or displacement sensor, or the like, coupled with the controller. Alternatively, the controller may initiate regular periodic muscle contraction per a pacing signal. In an exemplary embodiment, the structure comprises an eyelid implant, the electrode comprising an orbicularis oculi stimulation electrode. The controller can be configured to electrochemically modulate blinking of an eye of the patient.

In another embodiment, the invention provides a method for modulating contraction in a muscle of a patient, the method comprises transmitting a chemical agent from a reservoir implanted in the patient toward the muscle, and stimulating the muscle by energizing an electrode implanted in the patient adjacent the muscle, the chemical agent enhancing the stimulation.

In exemplary embodiments, the chemical agent and stimulation of the muscle electrochemically induce an effective blink of an eye of the patient. The chemical agent transmitted to induce the blink will often comprise less than 200 μl of acetylcholine or an equivalent quantity of carbachol, more often being less than 10 μl so as to provide a more comfortable implantable device size. The electrode can be energized with a potential of less than 0.5 volts for less than 200 msec to induce the blink, and electrodes along opposed major surfaces of first and second implanted structures (disposed in the upper and lower eyelids, respectively) may be energized. The electrodes often extend along the orbicularis oculi for a significant portion of a corresponding surface dimension of that muscle.

A bolus of the chemical agent will often be pulsed from at least one orifice of an implanted structure containing the reservoir, with each bolus being associated with a muscle contraction cycle. Another chemical agent may also be released from the structure to inhibit or counteract atrophy of the muscle. Encapsulation near the agent delivery site may be inhibited or its effects limited by delivering the agent through a protruding orifice, by eluting an antiproliferative agent, by applying a charge to a fluid transmission surface, by pulsing the agent with sufficient pressure, or the like. When the chemical agent is implanted into the patient more than two weeks before any cycle of muscle stimulation with that agent, the chemical agent may comprise carbachol.

In another aspect, the invention provides a hybrid device for stimulating contraction of a muscle of a patient body. The device comprises an implantable structure having chemical agent delivery means for transmitting a chemical agent to a muscle when the implanted structure is implanted in the patient, and electromagnetic stimulation means for directing electromagnetic stimulation toward the muscle. The chemical agent transmission means may optionally include a microfluidic system and/or microelectromechanical system (MEMS).

In another aspect, the invention provides a use of carbachol. The use comprises introducing the carbachol into a patient having a muscle, storing at least a portion of the carbachol in a reservoir within the patient, and transmitting at least a portion of the stored carbachol to the muscle so as to facilitate modulation of contraction of the muscle.

### BRIEF DESCRIPTION OF THE DRAWINGS

FIG. 1 illustrates a motor neuron comprising a cell body and at least one axon extending therefrom.

FIG. 2 illustrates an axon of FIG. 1 extending into a surface of a muscle fiber.

FIG. 3 provides a schematic illustration of a synapse.

FIG. 4 illustrates an embodiment of a delivery device of the present invention comprising a structure having a plurality of orifices.

FIG. 5 illustrates an embodiment of a delivery device having reservoirs and microfluidic channels.

FIG. 6 is a cross-sectional illustration of one embodiment of a delivery device of the present invention.

FIG. 7 illustrates an embodiment of a delivery device which may provide both chemical and electrical stimulation.

FIGS. 8A-8B illustrate embodiments of delivery devices which provide electrical stimulation.

FIGS. 9A-9C illustrate a method of placing an embodiment of a delivery device in proximity to the orbicularis oculi muscle.

FIG. 10 illustrates a pretarsal placement of the delivery device.

FIG. 11 illustrates a septal placement of the delivery device.

FIG. 12 illustrates, example locations for placement of delivery devices.

FIG. 13 illustrates access lines extending from a structure to a remote location.

FIG. 14 provides a view behind a patient's ear illustrating a controlling device.

FIGS. 15-16 provide schematic illustrations of embodiments systems of the present invention.

FIG. 17 illustrates devices of the present invention implanted in cheeks of a patient.

FIG. 18 schematically illustrates additional aspects of an exemplary electrochemical stimulation system in which different chemicals are used to pre-treat muscles to counteract atrophy, to stimulate muscle tissue, and to inhibit encapsulation of or ingrowth into the implanted device(s).

FIG. 19 is a functional block diagram schematically illustrating additional and/or optional structural components and interactions of the system of FIG. 18.

FIG. 20 is a functional block diagram schematically illustrating additional details of an electrochemical muscle modulation system similar to those of FIGS. 19 and 20.

FIG. 21 schematically illustrates use of an electrochemical muscle stimulation implant as an artificial synapse for reanimating denervated muscles.

FIGS. 22, 22A, and 22B illustrate an exemplary muscle implant device for electrochemical modulation of muscle contraction to provide an effective blink of an eye of a patient.

FIG. 23 schematically illustrates microfluidic device fabrication techniques that can be used to produce many embodiments of the implantable electrochemical muscle modulation devices described herein.

FIG. 24 illustrates a hydrolytically actuated microfluidic valve for optional use in the systems of FIGS. 18-20.

FIGS. 25 and 25A-25D illustrate alternative chemical delivery surfaces having microfluidic orifices disposed on protrusions or needles, for use in the systems of FIGS. 18-20.

FIG. 26 illustrates an alternative chemical agent delivery structure that may be modified for use in embodiments of the present invention.

FIGS. 27 and 28 illustrate alternative microfluidic channel network components which may be included in embodiments of implantable electrochemical muscle modulation devices, and representative performance of such components.

FIG. 29 illustrates alternative microfluidic pump structure types that might be used in embodiments of the invention.

FIGS. 30A-30F illustrate operation of alternative pumps that may be included in alternative embodiments to release and/or eject chemical agents.

FIGS. 30G-30M illustrate still further alternative electroosmotic pumps that may be included to release and/or eject chemical agents.

FIG. 31 illustrates a simple exemplary embodiment of a chemical delivery device.

FIG. 32 schematically illustrates electrical and control components of an embodiment of an muscle stimulation system.

FIGS. 32A and 32B illustrate top and side view of an implant having muscle stimulation electrodes for use in the system of FIG. 32.

FIG. 33 illustrates a user interface of a computer used as a controller in the system of FIG. 32.

FIGS. 34 and 35 illustrate an exemplary electrical stimulation signal waveform and muscle response.

FIGS. 36A-36C illustrate alternative exemplary implantable stimulation structures, and a stimulation structure implanted in an eyelid of a rabbit mode.

FIG. 37 is a flow chart schematically illustrating an exemplary method for treating a muscular denervation or other dysfunction.

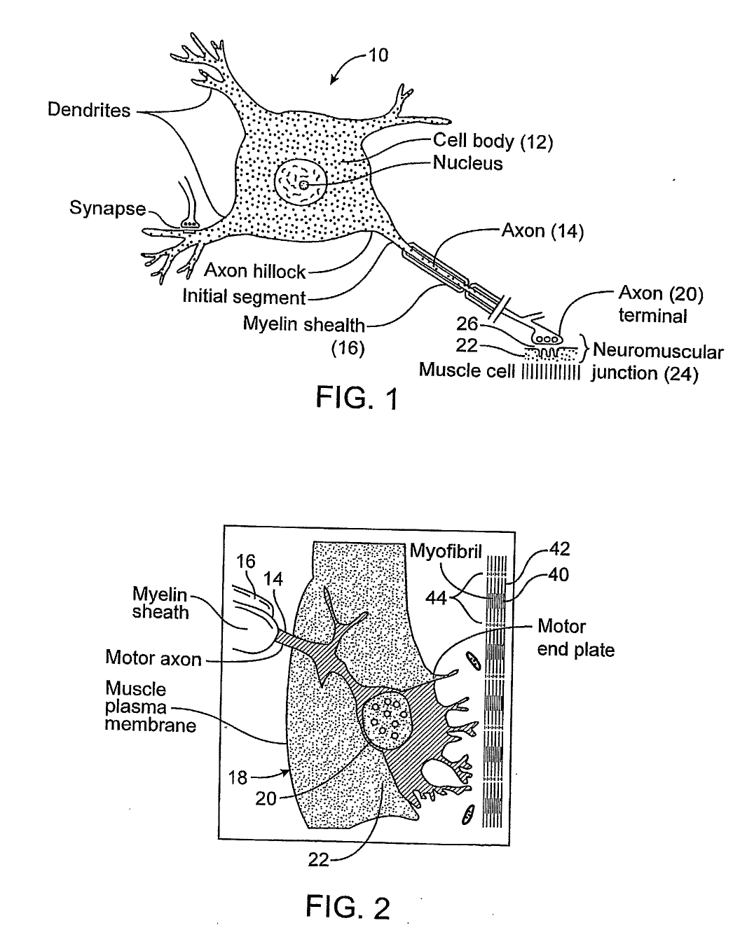

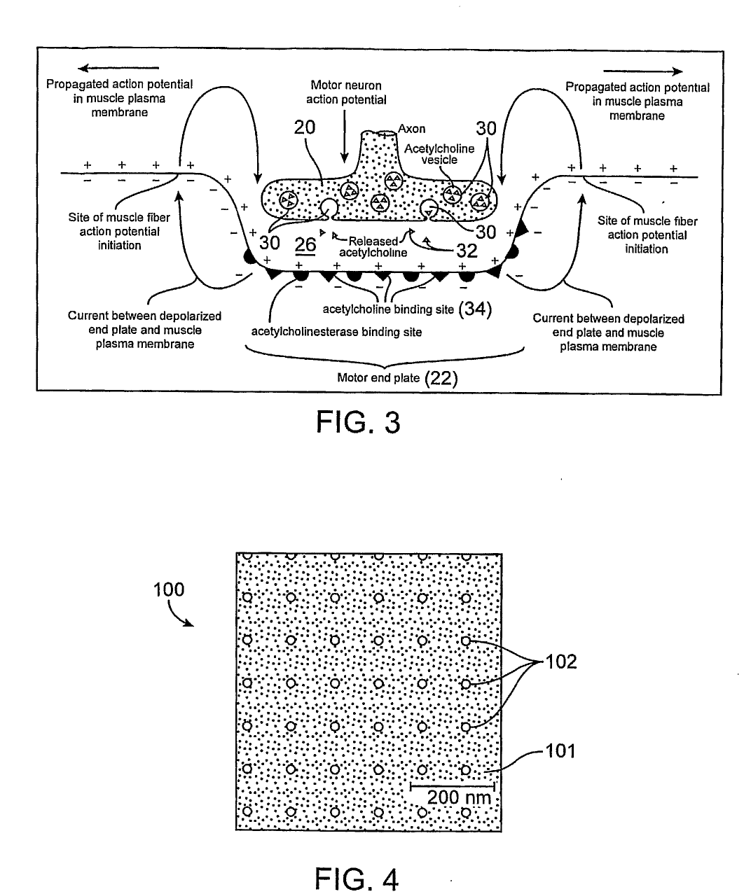

### DETAILED DESCRIPTION OF THE INVENTION

Embodiments of the present invention provide devices, systems, and methods for stimulating of tissue, often for stimulation of contraction, growth, and/or improved tone in muscle tissue. Facial and other muscle contraction may be modulated without the discomfort and pain that could result from electrode-only techniques, significantly improving quality of life for individuals having a wide variety of muscular dysfunctions. Embodiments employing hybrid implantable chemical and electromagnetic stimulation devices, systems, and methods may be particularly beneficial.

While exemplary embodiments will herein primarily be described with reference to treatment of muscles associated with the eyelids so as to allow a patient to blink, a wide variety of alternative embodiments may also be provided. Reanimation of any of a variety of facial muscles may be provided, including muscles of the lower face. Treatments of appropriate muscles may also enhance appearance, use, position, or comfort of a hand, arm, leg, foot, or the like. For example, treatment of associated muscles may alleviate foot drop. Treatments of muscles associated with the vocal chords may improve verbal communication, while treatments of muscles of the soft palate and/or tongue may alleviate snoring, sleep apnea, and other sleeping disorders. Patients suffering from degenerative nerve disorders such as amyotrophic lateral sclerosis (ALS) may be treated to enhance muscular control of (for example) the neck, helping to improve the quality of life and limit mortality. Nerve damage associated with trauma may be alleviated, and the treatments described herein may be combined with facial (and other) reconstructions to allow reanimation of facial tissues, or with transplantation techniques to allow reanimation of facial (and other) transplants, and the like. Treatments directed to muscles of a sphincter may provide controlled continence by improving muscle tone and/or by sealing the sphincter and allowing luminal flow when desired, facilitating treatment of urinary or fecal incontinence, gastro-esophageal reflux disease (GERD), or the like. A variety of atrophied muscles in the body can be stimulated to improve muscle tone and function. Broadly, the techniques set forth herein can sense or determine a biological need for muscle stimulation, and can effect stimulation in response to that need. Hence, the devices and techniques described herein may be used to treat a wide range of medical indications.

Many embodiments may be used to treat denervated muscles and/or pathologies of the neural system that degrade patient control over some or all of the muscles of the body. Embodiments may modulate movement of one member of a bilateral muscle pair based on corresponding contralateral muscle movements or commands. Hence, patients with unilateral vocal chord failure, unilateral facial paralysis, impaired control over the viewing axis movements of one eye, or the like, may benefit. Other embodiments may periodically modulate muscle contraction (for example, to enhance muscle tone), or may modulate contraction at least in part in response to an input command (typically from the patient) so as to produce a commanded tissue movement. Still other embodiments may determine that muscle contraction is appropriate based on external measurements or sensors, such as by monitoring sound from a patient having a sleep disorder. More generally, muscles may be treated to limit undesired contraction, to effect a desired movement, or to improve tone. Advantageously, denervated muscles that might otherwise atrophy (or have already atrophied) may be treated to enable induced muscular contraction, and then may be stimulated to effect controlled muscular contraction, thereby alleviating the effects of trauma and disease. The hybrid implant or chip systems described herein can utilize a combination of stimulation properties depending on the muscle group of interest. Options include electrochemical, electromagnetic and a wide variety of three stimulation options with the emphasis on the specific subtype shifting based on physiologic need (electrical-chemical-magnetic). The hybrid chip system may detect biologic need and respond with appropriate delivery combinations designed to meet the needs of the denervated, hypotonic or dysfunctional muscle, sphincter, or the like.

### I. Delivery Device

Delivery devices of the present invention typically comprise a structure 101 having a plurality of reservoirs, each reservoir holding a chemical agent which is released, such as ejected, through an orifice directly to a dysfunctional muscle. FIG. 4 illustrates an embodiment of a delivery device 100 comprising a structure 101 having a plurality of orifices 102 through which chemical agents are released. In preferred embodiments, the structure 101 is formed by micromachining, microelectromechanical systems (MEMS) technology, nanofabrication or other suitable methods. Processes and materials that may be used include, but are not limited to, imprint lithography, stamping, photolithography, thermal oxidation, dopant diffusion, ion implantation, LPCVD, PECVD, evaporation, sputtering, wet etching, plasma etching, reactive-ion etching, ion milling, silicon, silicon dioxide, silicon nitride, aluminum, anisotropic wet etching or single-crystal silicon, deep reactive-ion etching (DRIE), x-ray lithography, electroplating, low stress LPCVD films, thick film resist (SU-8), spin casting, micromolding, batch microassembly, piezoelectric films (such as PZT), magnetic films (such as Ni, Fe, Co, and rare earth alloys), high temperature materials (such as SiC and ceramics), mechanically robust aluminum alloys, stainless steel, platinum, gold, sheet glass, and plastics (such as polyvinyl chloride (PVC) and polydimethylsilicone or polydimethylsiloxane (PDMS)). Electrode or electrode patterns on the surface of delivery device 100 may be formed using lithographic and thin film deposition techniques used for semiconductor manufacturing, the electrode metal typically comprising a biocompatible metal such as gold, platinum, or titanium.

In particular, the structures 101 may be rigid, such as formed from a silicon wafer, or flexible, such as formed from PDMS. In some embodiments, a photosensitive substrate is layered onto the silicon wafer or PDMS. A microlithography patterned mask is then placed over the photoresist. The wafer or PDMS is subjected to ultraviolet light wherein the exposed areas of photoresist solubilize and are removed. Etching agents, such as acids or arsenic and other harsh chemicals, can be applied to remove the unprotected areas. Shapes and patterns dictated by the mask are thus formed into the wafer or PDMS. These shapes and patterns are designed as reservoirs and microfluidic channels within the structure 101. A polymer layer is then fused to the bottom of the silicon wafer or PDMS, sealing the reservoirs and channels within. Small orifices extend from the surface to the channels allowing chemical agents to flow in or out of the channels to the surrounding environment, such as to the dysfunctional muscle.

In other embodiments, the structure 101 is formed by spin casting polymers on microfabricated molds and cross-linking the polymers. PDMS is particularly suitable since it can be easily spun into thin layers and subsequently polymerized to produce a robust film. A thin layer of gold may be sputtered onto the microfabricated mold to reduce adhesion forces between the materials. The polymer is then separated from the mold which has created reservoirs and channels dictated by the mold. A polymer layer is then fused to the bottom of the silicon wafer or PDMS, sealing the reservoirs and channels within. Again, small orifices extend from the surface to the channels allowing chemical agents to flow in or out of the channels to the surrounding environment, such as to the dysfunctional muscle.

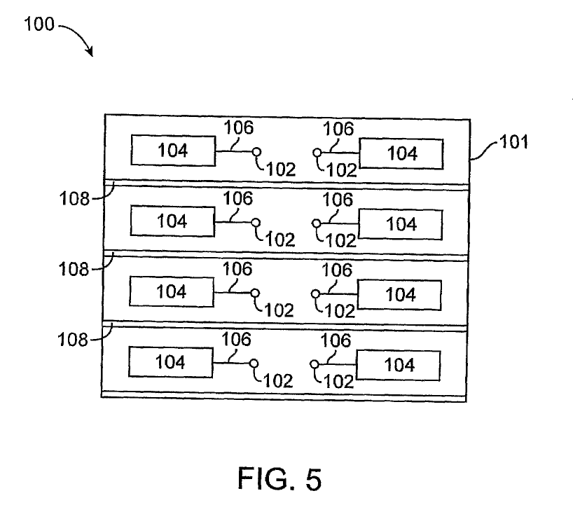

FIG. 5 illustrates an embodiment of a delivery device 100 revealing reservoirs 104 and microfluidic channels 106 therein. The channels 106 extend between a reservoir 104 and an orifice 102. A chemical agent within the reservoir 104 may transported through the channel 106 and out of the orifice 102 by electroosmotic actuation. Such actuation may be achieved with electrodes deposited within the device 100, such as within the channels 106 or adjacent to the orifices 102. In FIG. 5, electrodes 108 are illustrated as separating the reservoirs into rows, although a wide variety of alternative electrode patterns or structures might be employed. Electrodes may be comprised of any suitable material, such as metal, gold, platinum, etc. It may be appreciated that the structures 101 of the present invention may have any suitable size and thickness, such as 6 mm in length, 4 mm in height and 1 mm in thickness.

Due to the unstable nature of some chemical agents that may be utilized, embodiments may contain two or more chemicals, with the chemicals optionally being combined prior to and/or as they are released so as to form the appropriate chemical for muscle modulation. A system with two or more chemical reservoirs which would eject precise amounts of each substance simultaneously either into a channel or chamber for mixing just prior to ejection or into the surrounding tissue for mixing could be utilized. One example of this would be to use a very concentrated solution of chemical agent to be mixed with a diluting solution. In some embodiments, the diluting solution could be obtained from the body itself, such as from tears. A variety of suitable microfluidic processing structures have been described that may be employed for such chemical processing.

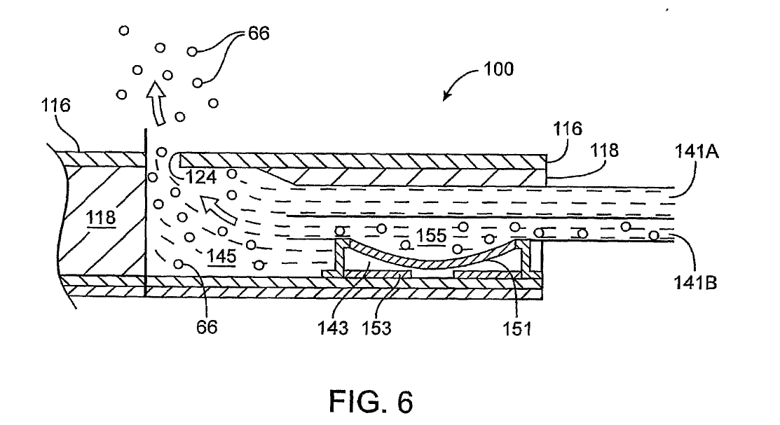

In some embodiments, the delivery device 100 may comprise components related to those described by Fishman et al. (US 2004/0224002), incorporated herein by reference for all purposes. One embodiment of the delivery device 110 is illustrated in FIG. 6 in a cross-sectional view. Here, an orifice or aperture 124 though a supporting layer 116 opens into channel 136 within an intermediate layer 118. A fluid conduit 141 carries a chemical agent 66 through the channel 145 and out through the orifice 124, with flow optionally induced by pump 143. The agent 66 is stored in a reservoir operably connected to pump 143 and microfluidic channel 145. In this embodiment, the fluid conduit 141 is comprised of two parts, a buffer inlet 141A and a transmitter inlet 141B. The pump 143 comprises a microelectromechanical (MEM) pump similar to those used in ink-jet printers to eject drops of fluid. Examples of such pumps are described in U.S. Pat. No. 5,734,395. A MEM pump includes a silicon diaphragm 151, a counter electrode 153 and a microfluidic channel 155 built over the diaphragm 151. Initially the diaphragm 151 is in an undetected configuration. The application of a minute bias voltage between the diaphragm 151 and the counter electrode 153 is effective to deflect the diaphragm 151 downward, as shown, thereby increasing the volume of the channel 55 region above the diaphragm 151 and drawing the chemical agent 66 from the reservoir. Removal of the bias voltage allows the diaphragm 151 to relax back to its initial position, forcing the chemical agent 66 out of the channel 55 and through the orifice 124.

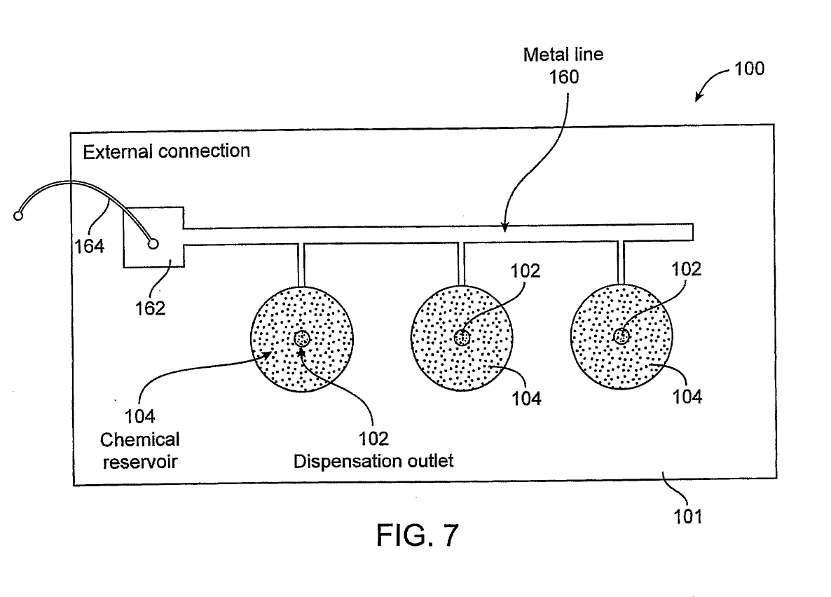

In other embodiments, the delivery device 100 provides chemical stimulation, by delivery of a chemical agent 66 such as described above, and electrical stimulation. FIG. 7 illustrates an embodiment of a delivery device 100 which may provide both chemical and electrical stimulation. Here, the device 100 includes a structure 101 having one or more metal lines 160 which can terminate in larger bond pads 162 and can extend outside of the body via thin wires 164 that are soldered or glued to the bond pads 162. The thin wires 164 are in turn connected to an external voltage signal source which supplies the electrical stimuli. The metal lines 160 may be further processed to incorporate microstructures designed for dispensation of the chemical agent 66 through orifices 102 from reservoirs 104 via electroosmosis or electrophoresis or mechanical pressure. The reservoirs 104 may be located within the structure 101 itself, or may be located separately and connected to the structure 101 via thin tubing.

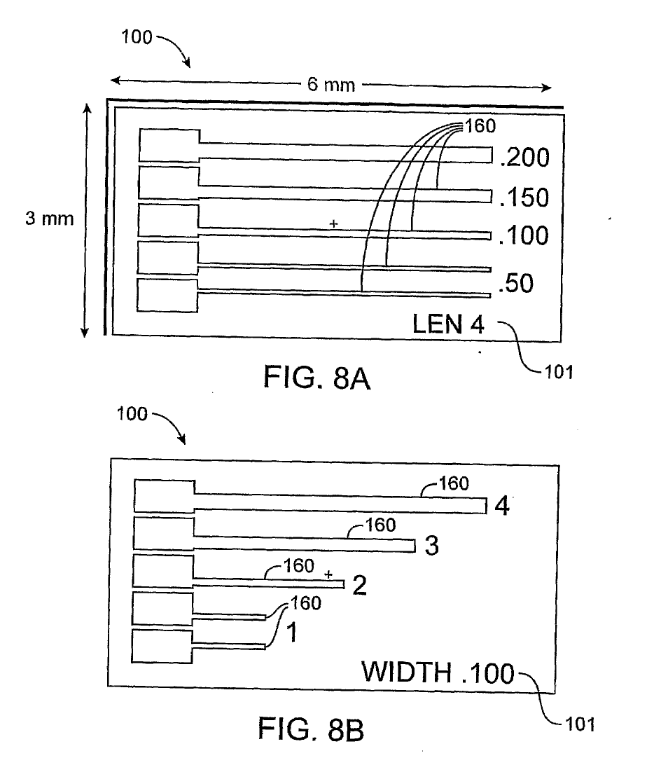

In other embodiments, the delivery device 100 provides electrical stimulation without chemical stimulation. FIGS. 8A-8B illustrate embodiments of delivery devices 100 which provides electrical stimulation. The devices 100 each comprise a structure 101 having a plurality of metal lines 160, each metal line 160 terminating in a larger bond pads 162. As described above, thin wires are soldered or glued to the bond pads 162. The thin wires 164 are in turn connected to an external voltage signal source which supplies the electrical stimuli. FIG. 8A illustrates metal lines 160 having a variety of widths. FIG. 8B illustrates metal lines 160 having a variety of lengths.

### II. Direct Stimulation of Facial Muscles

The facial nerve, cranial nerve seven (CN-7) exits the skull and courses down the jaw, diverging into a variety of branches which innervate facial muscles and other end organs. The major function of CN-7 is to supply motor innervation to the muscles of facial expression, allowing a person to blink, squint their eyes, raise their eyebrows, smile, and communicate emotion, to name a few. CN-7 dysfunction is a common problem affecting all races, both genders and all ages. It can be caused by inflammation, infection, stroke, cancer or following surgery or trauma. In the periocular region, CN-7 innervates the orbicularis oculi muscle to control closure of the eyelids. Opening of the eyelids, on the other hand, is controlled by the levator (innervated by the third cranial nerve) and Meuller's muscle (innervated by the sympathetic nerve). Thus, the three muscles involved in the production of synchronous eyelid opening and closing include the levator, Mueller's and orbicularis oculi muscles. As is the case throughout the body, when the eyelid is closed, the orbicularis oculi is stimulated and the levator/Mueller's muscles are inhibited. Failure to inhibit the antagonist muscles will prevent eyelid closure. Of note, simultaneous stimulation of the agonist and antagonist will result in spastic twitching without eyelid closure.

Closure of the eyelids is achieved by contraction of the orbicularis oculi muscle, a single oval sheet of muscle extending from the regions of the forehead and face and surrounding the orbit into the eyelids. When the orbicularis oculi muscle is denervated, dysfunction results in an inability to close the eyelid, ocular irritation, corneal breakdown, visual disability and pain. Orbicularis oculi tone and blinking ability is also beneficial for normal tear drainage into the nose.

Current conventional therapy of orbicularis oculi muscle dysfunction includes the application of thick ointments, frequent artificial tears and moisture chambers. This commonly results in severe visual blurring and incomplete relief of symptoms. Surgical treatments, such as tarsorrhaphy wherein the eyelids are partially sewn together to narrow the opening, are deforming and limit vision. Lateral tightening procedures for the lower eyelid and brow lifts merely pull tissues tighter and create additional disabilities and deformity. Other surgical treatments, such as the implantation of a gold or platinum weight (1-2 grams) or spring in the eyelids, are not effective in restoring tone and blink. Such weights rely on gravity to close the eyelid and are only functional in an upright position.

The structures and methods described herein may optionally make use of aspects of other nerve stimulation devices currently in use or now being developed. For example, the Synchrony Plus system may be available from Medtronic for conditions related to pain control. Related devices for vagus nerve stimulation may be commercially available from Cyberonics Inc., of Houston and others. Embodiments may also make use of aspects of commercially available external devices, such as the EMS-1C™ and EMS-2C™ electrical stimulators, which may be used for stimulation of muscle contraction in paralyzed muscle to slow muscle atrophy. U.S. Pat. No. 6,051,017 to Loeb, et. al., the full disclosure of which is incorporated herein by reference, describes an implantable microstimulator and related systems. Related implantable Bion devices may be under development by the Alfred P. Mann Foundation of Santa Clarita Calif. for nerve stimulation, and aspects of these devices and systems may also be employed in embodiments of the systems and methods described herein.

Effective eyelid reanimation may generally benefit from extremely low stimulation voltages; fast response times (20 msec from initial depolarization to eyelid closure), and relatively large volume delivery to a relatively large surface of the orbicularis oculi. Regarding stimulation voltages, when electrical stimulation alone is applied even the maximum electrical stimulation to the eyelids that may be tolerated by patients with facial nerve palsy may not result in an eyelid blink in some embodiments. Furthermore, the thin tissues around the eye may be sensitive to the unpleasant sensation created by significantly smaller (30 mAmp) levels of electrical stimulation. Hence, some other muscle activation (in place of electrical stimulation alone or in combination with electrical stimulation) may be employed. Utilization of an electrochemical stimulation system may lower the current for functional stimulation, as the muscle is partially stimulated with the use of neurotransmitters, which produces a more comfortable stimulation experience. Denervated muscle stimulation may generally employ pulse widths of at least 10 msec for successful stimulation.

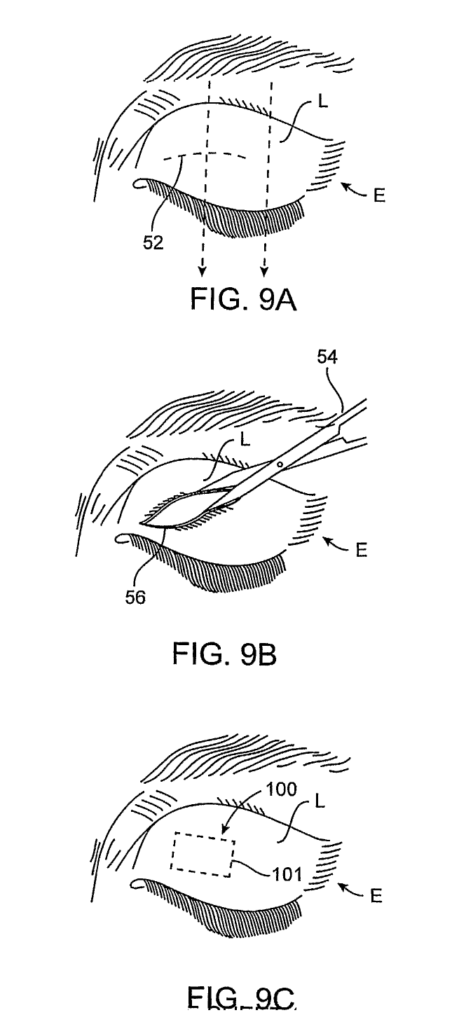

The delivery device of the present invention may be used to restore eyelid blinking in patients with seventh nerve palsies. The delivery device stimulates and paces the orbicularis oculi muscle in a fashion that will mimic the natural chemical stimulation of the orbicularis oculi muscle. This may restore resting tone, spontaneous blink and/or voluntary blink of the eyelids. FIGS. 9A-9C illustrate a method of placing an embodiment of a delivery device 100 of the present invention in proximity to the orbicularis oculi muscle. Options for placement, using the same incision site, include pretarsal, preseptal and orbital orbicularis oculi placement. In preferred embodiments, the delivery device 100 is comprised of a structure 101 which is positioned over the orbital component of the orbicularis oculi as this placement maximizes the distance from the levator muscle—the antagonist muscle to the orbicularis—thereby minimizing any inadvertent simultaneous stimulation of both muscles which may cause twitching without eyelid closure. FIG. 9A illustrates an eye E having an eyelid L in a closed position. Incision line 52 across the eyelid L indicates a possible location for accessing the orbicularis oculi muscle. FIG. 9B illustrates opening of the incision 52 with an instrument 54 revealing a pocket 56 that directly overlies the orbicularis oculi muscle. The delivery device 100 is then inserted into the pocket 56, as illustrated in FIG. 9C. The orbicularis oculi is then closed with buried vertical mattress sutures, preferably 6-0 vicryl sutures. The incision 52 of the eyelid L is then sutured closed.

The delivery device 100 may be positioned in a variety of locations to stimulate the orbicularis oculi muscle. Optimum location may be determined by mapping of the orbicularis oculi to determine optimum stimulation parameters. This may be particularly desired because the anatomy and physiology of the orbicularis oculi muscle is relatively unique in the body. For example, the nerve to muscle ratio is the most abundant in the body (approximately 1:3). Also, the tissues are extremely well vascularized allowing abundant oxygenation and effective toxin removal. In addition, the motor units have an unusual “grape-like” morphology. Further, the facial muscles and extraocular muscles have the shortest contraction time in the body (7 msec) and the highest potential frequency of contraction (number of events per second). The resting tone of the orbicularis, for example, has a contraction frequency of 50 contractions per second which rises to 170 contractions per second. This high potential frequency of contraction combined with the low voltage system of the orbicularis oculi muscle allows rapid, fine and sustained orbicularis oculi movements that exceed the characteristics of other skeletal muscles.

In preferred embodiments, the device 100 location is over the pretarsal and preseptal component of the orbicularis. The orbicularis oculi has the smallest myofibril structures in the body. 80-90% is type 2 (slow twitch) myofibrils, this approaches 100% in the pretarsal region of the muscle. The myofibrils are variable in size with the pretarsal ones 36% of the length of those found in the preseptal region. Studies on rabbit and human eyelids demonstrated a very similar distribution of neuromuscular junctions (Lander, 1994). Multiple innervation is unusual, rather a single NMJ is typically located in the middle third of the myofibril. The NMJ clusters are spread through the pretarsal orbicularis. In contrast, the NMJ are grouped in the medial and lateral canthal regions of the preseptal orbicularis. It is desired to produce as natural a spontaneous blink as possible and also to provide a mechanism for voluntary closure. The pretarsal and preseptal parts of the orbicularis oculi muscle are responsible for the spontaneous blink. The orbital portion functions in voluntary closure. The delivery device 100 may control spontaneous blinking by eliciting preset timed electrical stimuli for closure. Alternately, a connection could be made from the contralateral orbicularis oculi to trigger symmetric closure of the eyelids.

In order to ensure simultaneous contraction of all the pretarsal and/or preseptal fibers, which would be desirable to stimulate a functional blink action of the orbicularis oculi, the electrodes may be sized and oriented such that they can span the entire length of the pretarsal and/or preseptal orbicularis oculi. Implants would preferably be placed in the upper and lower eyelid, to capture all the relevant muscle fibers during stimulation.

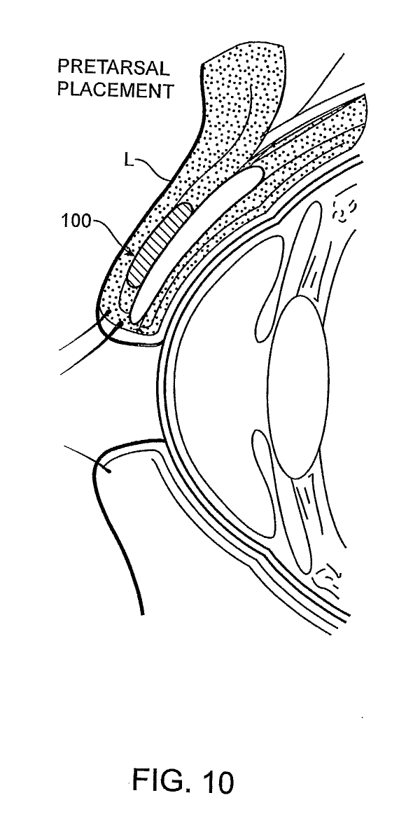

FIG. 10 illustrates pretarsal placement of the delivery device 100. The device 100 is shown placed behind the skin and subcutaneous fat, overlying the pretarsal orbicularis oculi muscle, which overlays the tarsal plate. Here, the pocket 56 includes the pretarsal space. The levator aponeurosis is stripped from its attachments to tarsus in the area of planned implantation, thus baring the anterior tarsal surface and effecting a modest levator recession. The delivery device 100 is centered over the bare superior tarsal surface. The orbicularis oculi is closed over the device 100 with interrupted sutures and the skin is closed with a running suture.

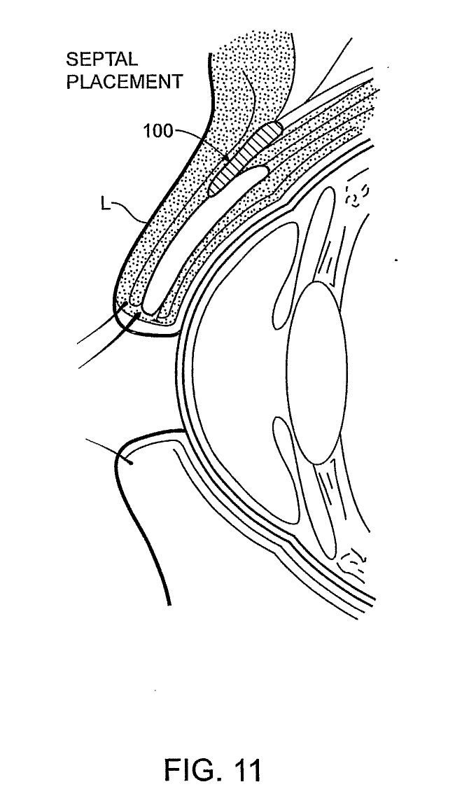

FIG. 11 illustrates a septal placement of the delivery device 100. The device 100 is shown placed behind the skin and subcutaneous fat, overlying the pretarsal orbicularis oculi muscle. Here, the pocket 56 is created to make room for the device 100 on the surface of the orbital septum and the tarsal plate. The device 100 is tied to the orbital septum with a single suture to hold it in place until the tissues heal around it and through the suture holes. The orbicularis oculi is closed over the implant with interrupted sutures and the skin is closed with a running suture.

In other embodiments, the delivery device 100 is implanted in the preseptal or pretarsal lower eyelid, or underneath the skin just lateral to the eye.

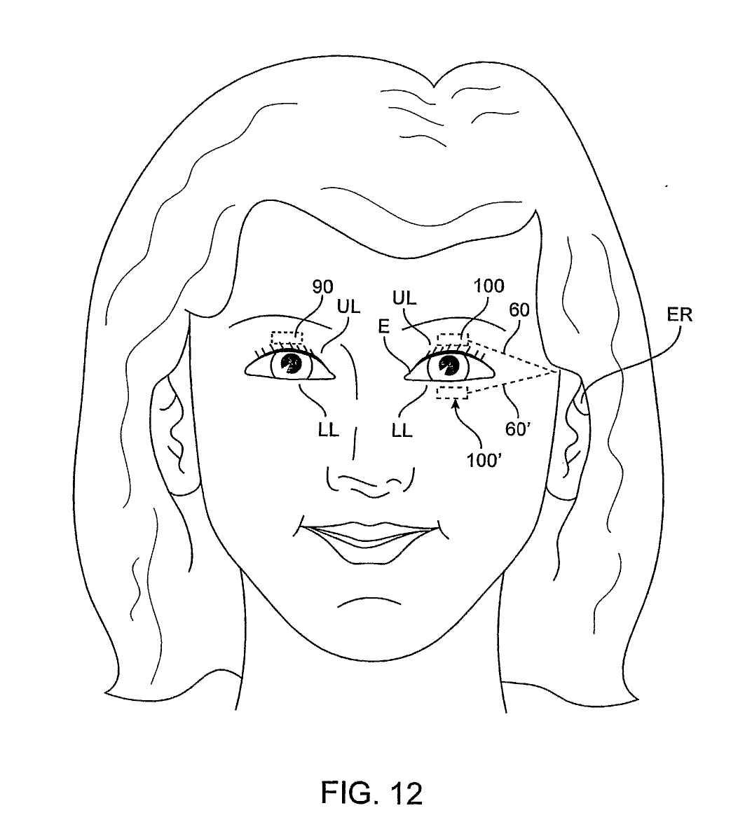

FIG. 12 illustrates, among other features, example locations for placement of delivery devices 100, 100′. Here, a delivery device 100 is shown positioned within the upper eyelid UL of a patient's left eye. Another delivery device 100′ is also shown positioned within the lower eyelid LL of the patient's left eye. Since blinking may be triggered by stimulation of the upper eyelid UL or lower eyelid LL, only one of the delivery devices 100, 100′ may be present. However, it may be appreciated that the presence of both delivery devices 100, 100′ may be desired as simultaneous stimulation at multiple locations in the muscle enables the production of blink with less current, providing a more comfortable action of the device.

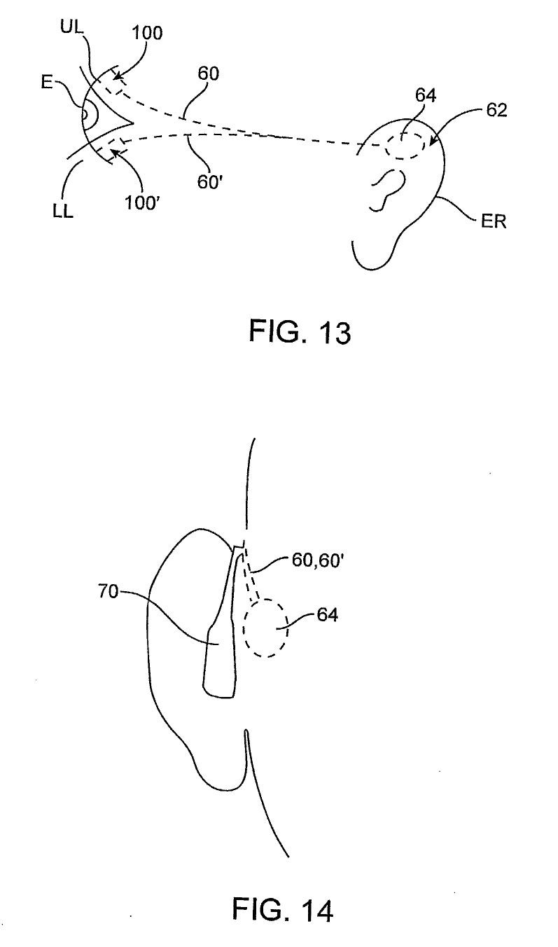

When delivery devices 100, 100′ are implanted in locations such as the eyelids UL, LL or other visible areas of the face, it may be desired to access the delivery devices 100, 100′ (such as to refill the reservoirs, provide electrical input, etc.) via a remote location. Thus, each delivery device 100, 100′ may include an access line 60, 60′ which extends to a remote location 62, such as behind the ear ER, as illustrated in FIG. 13. The access lines 60, 60′ may independently extend to the remote location 62 or may join together so that a single access line continues to extend to the remote location 62. In some embodiments, the access lines 60, 60′ are connected with a main reservoir 64 which resides within the remote location 62. The main reservoir 64 feeds the individual reservoirs within the delivery devices 100, 100′ via the access lines 60, 60′. Thus, the main reservoir 64 holds a chemical agent 66, such as acetylcholine 32. If more than one chemical agent 66 is desired to be delivered from the delivery devices 100, 100′ additional main reservoirs 64 may be present, each containing the desired chemical agent and each connected with the appropriate reservoirs within the delivery devices. The main reservoir 64 may be implanted within the remote location 62 so that refilling of the main reservoir 64 is achieved by, for example, injecting the chemical agent 66 through the skin and into the main reservoir 64. In such instances, the main reservoir 64 may be comprised of a silicone bladder to allow resealing of the reservoir 64 after each injection. In other embodiments, the main reservoir 64 resides externally to the patient so that the access lines 60, 60′ extend through the skin. It may be appreciated that the remote location 62 may reside at any distance from the delivery devices, including adjacent to the delivery devices.

FIG. 14 provides a view behind the ear ER of the patient of FIG. 12. In this embodiment, the access lines 60, 60′ extend to the main reservoir 64 which is implanted beneath the skin, as indicated by dashed line, behind the ear ER. Thus, the main reservoir 64 may be refilled by injection through the skin. The patient also wears a controlling device 70 external to the body, such as behind the ear ER as shown in FIG. 14. The controlling device 70 provides control signals to the delivery devices 100, 100′. Typically, the control signals are provided by wireless transmission.

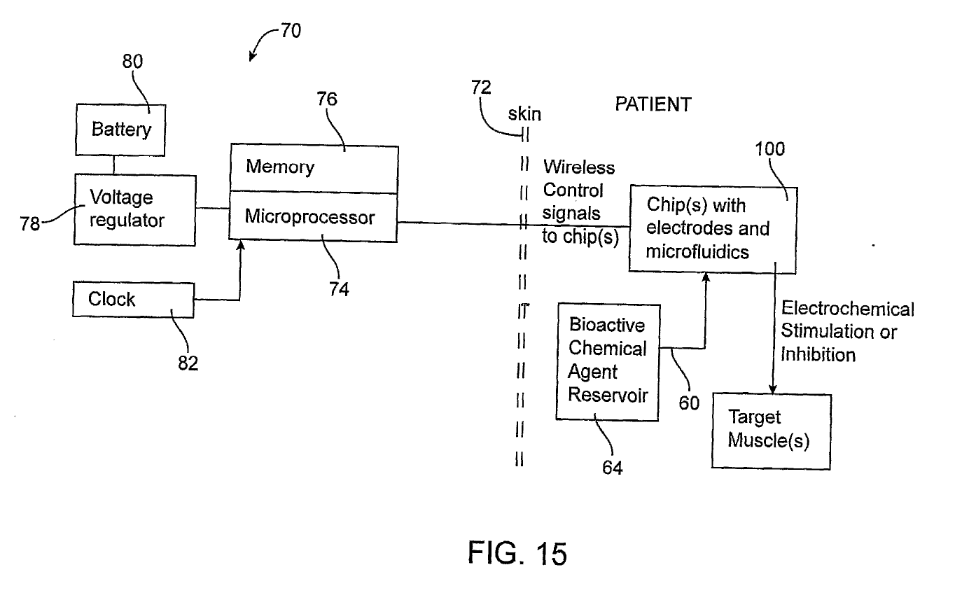

FIG. 15 provides a schematic illustration of an embodiment of a system of the present invention. Here, the controlling device 70 resides external to the skin barrier 72, and the delivery device 100, access line 60 and main reservoir 64 reside internal to skin barrier 72. In this embodiment, the controlling device 70 includes a microprocessor 74, memory 76, a voltage regulator 78, a battery 80, and a clock 82. In a preferred embodiment, the microprocessor 74 is designed for low power systems in order to maximize battery life. A software program resides on the system memory 76 that will work with the microprocessor 74 to coordinate sensor data processing, actuation of the various devices, system monitoring and calibration. The memory 76 will have the capability to be re-written to allow the software program to be updated periodically. A voltage regulator 78 will be used to condition the battery voltage to the desired value to drive the microprocessor 74 and other electronic components. The clock 82 provides timing input to the microprocessor 74 so that commands can be run at precisely timed intervals. Thus, the delivery device 100 may cause the patient to blink in pre-set timed intervals by eluting the chemical agent in controlled intervals.

In order to provide the appearance of natural blinking, it is desired that both of the patient's eyes blink symmetrically or in unison. Thus, the blinking of the impaired eye may be synchronized with the blinking of the contralateral unimpaired eye. In some embodiments, this is achieved with the use of a sensing device 90 which is implanted near the unimpaired eye, as illustrated in FIG. 12. In particular, the sensing device 90 is implanted in a location so as to sense the contractions of the functional orbicularis oculi muscle.

Sensing may be accomplished by the detection of changes in voltage or movement at the contralateral synchronizing muscle. Sensors composed of electrodes may sense voltage changes. Pressure sensors or accelerometers, both of which can be microfabricated in a small form factor, can be used to sense muscle motion and provide electrical feedback signals. Alternatively, the signal can be triggered on a fixed interval after the release of acetylcholine.

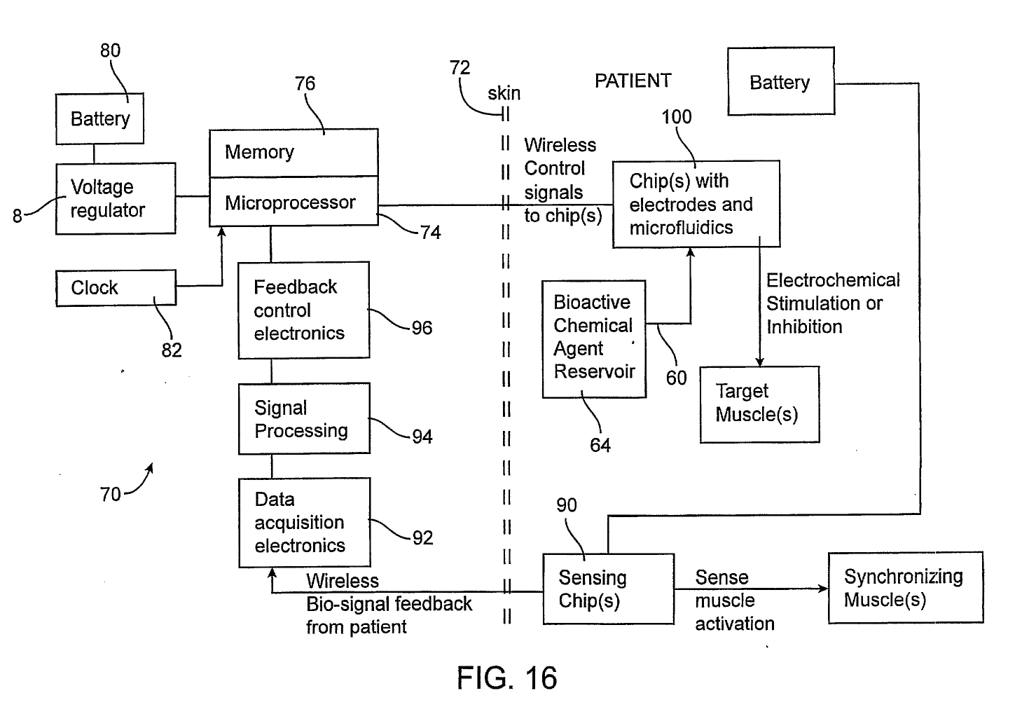

A feedback signal from the sensing device 90 is transmitted to the controlling device 70 as illustrated schematically in FIG. 16. As indicated, the controlling device 70 resides externally to the skin barrier 72 and includes data acquisition electronics 92 which receive the feedback signals from the sensing device 90. The feedback signals are processed by signal processing electronics 94 and feedback control electronics 96 which provide input to the microprocessor 74. The microprocessor 74 is then able to actuate the delivery device 100 based on feedback from the sensing device 90. Consequently, the delivery device 100 causes the patient to blink in a synchronized fashion by eluting the chemical agent in coordination with the blinking of the contralateral eye. The sensing device 90 may be powered by the same battery which powers the delivery device(s) 100. Subcutaneous wired connections may be fashioned from a single battery to all or any portion of the implanted devices 90, 100. The battery may be located either externally or subcutaneously. A battery located subcutaneously can be charged inductively, a technique commonly used in other battery-powered medical implants.

For substantially synchronized blinking of both eyes in response to blinking of a naturally functioning eye, sensing device 90 may be placed in (or otherwise be coupled to) the contralateral eyelid. Sensing device 90 may detects depolarization by sensing increased calcium concentrations, voltage alterations, and/or the like. Sensing device 90 may then, via a wire, a wireless communication link, a radio frequency system, or the like, communicate to the eyelid coupled to device 100, a signal from the sensing device preferably being communicated within 20 msec to provide effectively synchronous eyelid movement. The orbicularis oculi is then stimulated using acetylcholine and/or calcium to contract. Optionally, a 20 mm×10 mm×1 mm or smaller device 100 may be implanted in both the upper and lower eyelids. In other embodiments, related implantable devices may be placed in the brow, midface and/or peri-oral region. Pores in device 100 may also contain acetylcholinesterase to allow the muscle to be quickly returned to a state that is capable of rapid redelivery of ACH. The electrical source (both on the sensing side and paralytic side) and additional chemical reservoirs (paralytic side only) may be located subcutaneously behind the ears.

An external trigger may be fashioned, optionally located behind the ear, which enables voluntary on demand eye closure. This may be utilized during any time it is perceived that there may be a threat of a foreign body approaching the eye, such as sand, or any other perceived threats to the eye, such as extreme bright light.

In some patients, one or more eyelids lack resting tone causing the affected eyelid to sag or droop. Drooping lower eyelids may cause the eyelids to be unable to close leading to tearing, irritation, corneal breakdown and visual blurring, to name a few. Drooping upper eyelids may cause the eyelids to be unable to open leading to functional blindness. In addition, such conditions are visually distracting and unnatural in appearance. The delivery device 100 of the present invention may be used to provide resting tone to one or more eyelids. In some embodiments, this is achieved by the controlling device 70 actuating the delivery device 100 to provide a low level constant elution of a chemical agent 66, such as acetylcholine 32, from the device 100. This may be accomplished in other embodiments through the use of continuous low voltage electric stimulation to the target muscle(s).

The above described examples focus on facial muscles, particularly the orbicularis oculi muscle, which lack the natural ability to contract, either to cause movement or to provide resting tone. However, in some patients the facial muscles are overactive, contracting at undesired times or unceasingly contracting. Such conditions include blepharospasm, hemifacial spasm, ocular apraxia and superior oblique myokymia. The delivery device 100 of the present invention may also be used to treat such undesired contraction of muscles. Periodic slow release of a chemical agent, such as botulinum toxin, can be utilized to affect such an inhibition of unwanted muscle activity.

As mentioned above, the facial nerve, cranial nerve seven (CN-7) supplies motor innervation to a variety of muscles related to facial expression. Damage to the facial nerve may also cause facial drooping, disrupting speaking, eating and social interaction. Thus, the delivery device 100 of the present invention may also be used to stimulate muscles throughout the face which lack muscle tone or desired muscle control. FIG. 15 illustrates among other features, example locations for placement of delivery devices 100, 100′ along the left cheek in the vicinity of muscles desired to be stimulated. Examples of muscles of the face which may be appropriate for stimulation include the frontalis, the zygomaticus major, the levator anguli oris, and buccinator. Delivery devices 100 would be placed directly over the target muscle(s), ideally overlying the portion of the muscle with the highest concentration of neuromuscular junctions. Any number of delivery devices 100 may be used to stimulate a muscle, including one, two three, four, five or more, and any number of muscles may be stimulated.

Again, each delivery device 100, 100′ may include an access line 60, 60′ which extends to a remote location 62, such as behind the ear ER, as previously illustrated in FIG. 8. The access lines 60, 60′ may independently extend to the remote location 62 or may join together so that a single access line continues to extend to the remote location 62. In some embodiments, the access lines 60, 60′ are connected with a main reservoir 64 which resides within the remote location 62. The main reservoir 64 feeds the individual reservoirs within the delivery devices 100, 100′ via the access lines 60, 60′. Thus, the main reservoir 64 holds a chemical agent 66, such as acetylcholine 32. The main reservoir 64 thus functions as described above. Again, it may be appreciated that the remote location 62 may reside at any distance from the delivery devices, including adjacent to the delivery devices.

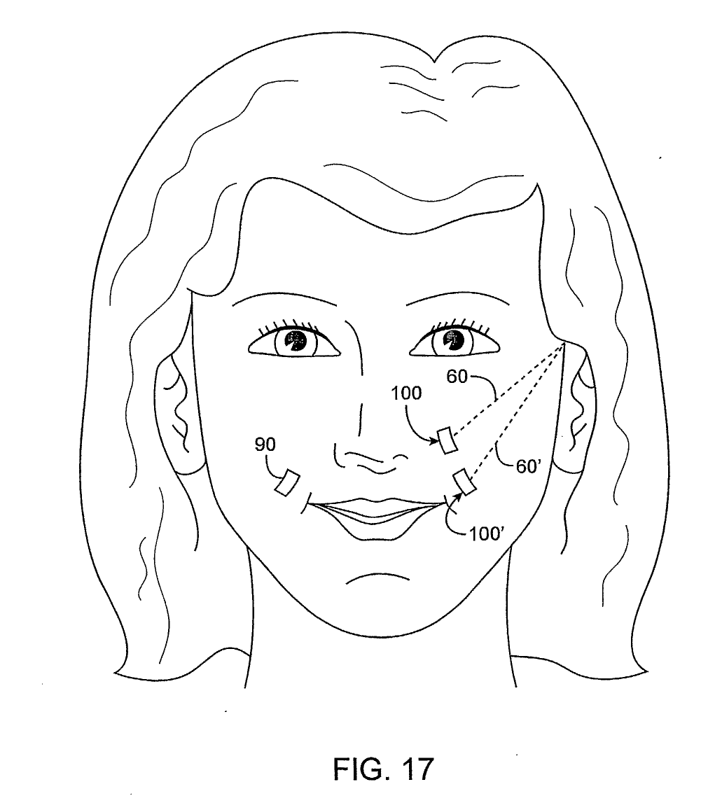

Typically, the features of the face generally contract symmetrically, the left side of the face contracting in unison with the right half of the face when smiling, frowning, etc. Thus, it is often desired that contraction of the impaired cheek is synchronized with contraction of the contralateral unimpaired cheek. In some embodiments, this is achieved with the use of a sensing device 90 which is implanted within the unimpaired right cheek, as illustrated in FIG. 17.

Sensing may be accomplished by the detection of changes in voltage or movement at the contralateral synchronizing muscle. Sensors composed of electrodes may sense voltage changes. Pressure sensors or accelerometers, both of which can be microfabricated in a small form factor, can be used to sense muscle motion and provide electrical feedback signals. Alternatively, the signal can be triggered on a fixed interval after the release of acetylcholine.

A feedback signal from the sensing device 90 is transmitted to the controlling device 70 as illustrated schematically in FIG. 16. As indicated, the controlling device 70 resides externally to the skin barrier 72 and includes data acquisition electronics 92 which receive the feedback signals from the sensing device 90. The feedback signals are processed by signal processing electronics 94 and feedback control electronics 96 which provide input to the microprocessor 74. The microprocessor 74 is then able to actuate the delivery device 100 based on feedback from the sensing device 90. Consequently, the delivery device 100 causes the patient's cheek to contract in a synchronized fashion by eluting the chemical agent in coordination with the contraction of the contralateral cheek. The sensing device 90 may be powered by the same battery which powers the delivery device(s) 100. Subcutaneous wired connections may be fashioned from a single battery to all or any portion of the implanted devices 90, 100. The battery may be located either externally or subcutaneously. A battery located subcutaneously can be charged inductively, a technique commonly used in other battery-powered medical implants. In alternative embodiments, the controlling device could be implanted under the skin.

An external trigger may be fashioned, perhaps located behind the ear, which enables voluntary on demand contraction.

As described above, the delivery device 100 of the present invention maybe used to provide resting tone to one or more muscles. In some embodiments, this is achieved by the controlling device 70 actuating the delivery device 100 to provide a low level constant elution of a chemical agent 66, such as acetylcholine 32, from the device 100. This may be accomplished in other embodiments through the use of continuous low voltage electric stimulation to the target muscle(s). This corrects general drooping and sagging of facial features.

### III. Direct Stimulation of Other Muscles

The delivery devices 100 of the present invention may be used to simulate other muscles in the body to treat a variety of other conditions. In these examples, the delivery device 100 is positioned on, near or within a target muscle for treatment in a manner similar to the methods described above in relation to the facial muscles. For example, the delivery devices may stimulate the extraocular muscles to control movement of the eyes. The orbicularis oculi may be stimulated to inhibition its unwanted hyperactivity in cases of blepharospasm. Vocal chord paralysis may be corrected by stimulation of the posterior cricoarytenoid muscle. Sleep Apnea may be corrected by stimulation of the genioglossus muscle. Diaphragm paralysis may be treated in patients with amyotropic lateral sclerosis. In addition, the systems, devices and methods of the present invention may be used in cardiac pacing, peripheral nerve damage, prostate cancer, and tonic bladder dysfunction, to name a few.

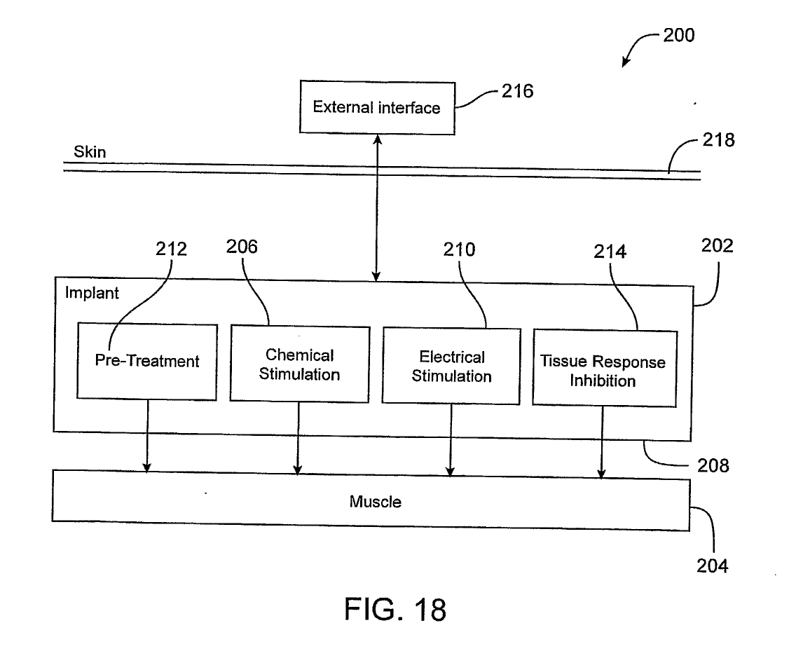

Referring now to FIG. 18, a simplified schematic of an embodiment of a muscle contraction modulation system 200 includes one or more implanted structure 202 (one shown here for simplicity) for treatment of a muscle 204. A chemical agent reservoir 206 within structure 202 contains a muscle stimulation agent, which is generally directed to muscle 204 through a fluid transmission surface 208. As indicated above, the agent may be ejected from one or more orifice of surface 208 using a microfluidic channel network of structure 202, the network having an appropriate pump powered by an implanted battery or the like.

Acetylcholine may be contained in reservoir 206 for stimulation of muscle contraction. Exemplary embodiments may use a commercially available injectable acetylcholine solution such as Miochol E™ (acetylcholine chloride), which is available from Novartis pharmaceuticals. Dilutions of 1-10 mg/ml may be released from surface 208, with the volume released for a contraction cycle often being in a range from about 0.5 nl to about 200 μl, more often being from about 0.5 nl to about 10 μl to provide a smaller, more comfortable implanted device volume. As the stable life of acetylcholine at body temperature may be limited, it will often be advantageous to use the acetylcholine from reservoir 206 within about two weeks or less, optionally within about 1 week or less. Advantageously, acetylcholinesterase (which may be produced by the patient or introduced by structure 202) provides a deactivator to limit the effects of the chemical stimulation provided by acetylcholine.

Where the chemical agent will remain within reservoir 206 for a significant period of time (such as more than two days, more than a week, more than two weeks, or even more than a month) before at least a portion of the agent is used to stimulate muscle 204, it may be advantageous to use a muscle stimulation analogue of acetylcholine having a longer stable life. A nicotinic mimetic may be used, optionally comprising carbachol (Ethanaminium, 2-[(aminocarbonyl)oxy]-N,N,N trimethyl-, chloride), such as that commercially available under the brand names Carbastat™, Carboptic™, Isopto Carbachol™, or Miostat™ from Alcon and other suppliers. In comparison to acetylcholine, carbachol may have a significantly greater life within reservoir 206, and may also remain active for a longer time when released from structure 202, due to the lack of a deactivator, such as seen in acetylcholinesterase. Hence, where carbachol will be released to stimulate each muscle contraction cycle, the quantity may be sufficiently low to diffuse or otherwise dissipate within the overall muscle stimulation cycle time.

Referring still to FIG. 18, structure 202 will preferably electrochemically stimulate contraction of muscle 204 through coordinated release of the chemical agent from reservoir 206 and electrical stimulation by a stimulation electrode 210. Electrode 210 will generally be disposed along or sufficiently near surface 208 for electrical stimulation of the same tissue exposed to the chemical agent, and the chemical agent can significantly reduce the amplitude and pulse width of the electrical stimulation signal used to produce a desired muscular contraction. Preferably, where a surface of muscle 204 engaged by surface 208 has a length, a corresponding length of electrode 210 will be at least a significant portion (such as at least 20%) of that muscle length, often being at least a majority of the muscle length, and ideally being almost all of the muscle length. The electrical signal applied by electrode 210 to the muscle will typically be less than 1.0 V, often being about 0.5 V or less, and will often have a pulse width (for each contraction of the muscle) of about 200 msec or less.

Chronically denervated and other dysfunctional muscles often atrophy. On histopathologic inspection fibrosis and fat infiltration may also be present. Fortunately, neuromuscular junction structure and function may remains intact with mild disorganization of the location of the receptors. Pre-treating muscle 204 with an appropriate agent 212 may help counteract or inhibit atrophy. Although embodiments of implantable structure 202 may include a dedicated reservoir (and/or other fluid delivery components) for pre-treatment agent 212, other systems may make use of the chemical stimulation reservoir 206, for example, initially introducing a pre-treatment agent in reservoir 206 and thereafter introducing a muscle stimulation agent. While sometimes referred to as “pre-treatment agents,” muscle trophic factors may be used before, during, or after muscle stimulation. Providing trophic factors to the muscles may be particularly beneficial in severely atrophic cases. Exemplary trophic agents include IGF-1 (insulin-like growth factor), which is structurally related to insulin and produced in response to growth hormone. IGF-1 will induce satellite cell recruitment which can result in muscle cell growth.

A tissue response inhibiting means such as an anti-encapsulation means 214 may be provided with implantable structure 202 to inhibit orifice overgrowth and the like. The natural response of the body to structure 202 will be to encapsulate the implanted structure, similar to what occurs with the gold weights that are now placed in an orbicularis pocket, and to the effects resulting from stents used throughout the body. Options to limit the detrimental effects of encapsulation of structure 202 include the use of microneedles that protrude out of the surface 208 through the fibrotic capsule. In some embodiments, a coating on surface 208 (optionally on or near the needle surfaces) with a slow release anti-inflammatory (similar to those of drug-eluting stents) may be employed. Suitable anti-encapsulation means 214 may comprise anti-inflammatory agents, anti-proliferative agents, chemotherapy drugs, anti-metabolites such as Fluorouracil (5FU), insulin-like growth factor (IGF-1), Mitomycin, and the like. Suitable coatings will often include these or other agents impregnated within a polymer matrix. Matrices for the coatings may be commercially available from SurModics, Inc. of Minnesota; Angiotech Pharmaceuticals of Canada, and other suppliers. Still further optional encapsulation inhibiting means 214 comprise circuitry and/or a material along surface 208 to present a charged surface that repels fibroblasts. Other options include generating a pressure head for chemical agents passing from reservoir 206 through surface 208 of about 2 psi or more, for example, to dislodge cells with each spritz. Mechanical anti-encapsulation means may also be provided, such as a rotor or reciprocating wiper that clears the orifice with each blink or at a prescribed interval (such as every 24 hours). An exemplary rotor structure may comprise a screw which rotates to effectively seal the orifice between chemical release, and which rotates to open the orifice.

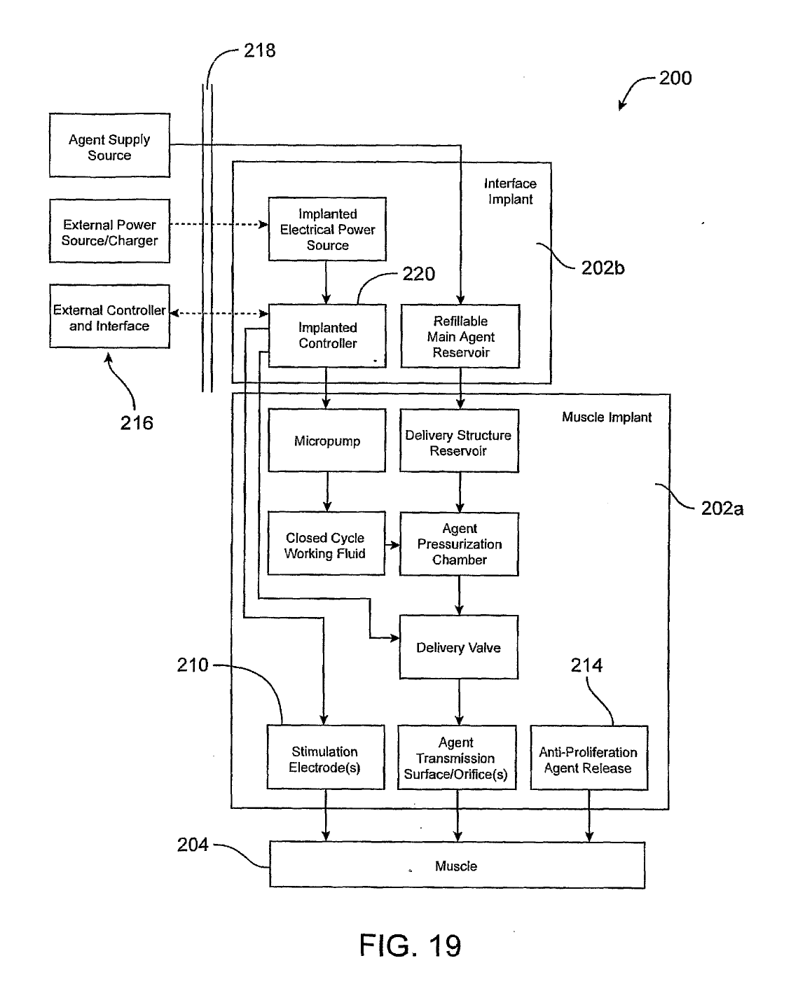

Referring now to FIGS. 18 and 19, a variety of structures may be used as an external interface 216, allowing the passage through skin 218 of command data to the implanted structure 202, fluids to and/or from structure 202 to re-supply chemical agents or remove waste fluids, feedback, system diagnostic, and internal telemetry data, and the like. As generally indicated above, implanted structure 202 may comprise a first implanted structure or synapse chip 202a implanted with surface 208 engaging muscle 204, and a second implanted structure 202b implanted at a convenient location for interfacing with external components of the system, such as a battery charger, agent injection syringe, data interface, and the like. A plurality of muscle implant structures may by associated with each implanted interface structure. One or more of the implanted structures may comprise a substrate having components of a microfluidic network for release of chemical agents, a digital signal processor for controlling the release of chemical agents and the application of electrical stimulation, a wireless transceiver, and/or the like. The controller 220 may, at least in part, reside in an external processor, on one or more implanted synapse chip structure 202a engaging the muscle, on one or more interface structures 202b, and/or on a separate dedicated implant in any of a wide variety of alternative fluidic and data system architectures. Controller 220 will typically comprise reprogrammable data processing hardware and/or software, with the software often being in the form of machine-readable programming code or instructions for implementing the methods described herein. The code may be embodied in a tangible media such as a memory, a magnetic or optical recording media, or the like. The code and associated data may be transmitted by electrical signals (along wires) by optical signals, and/or using wireless transmission technologies similar to those used in the Bion™ stimulation device. Exemplary fluid flow components, electrical stimulation components, and control components of system 200 may be more fully understood when described separately.

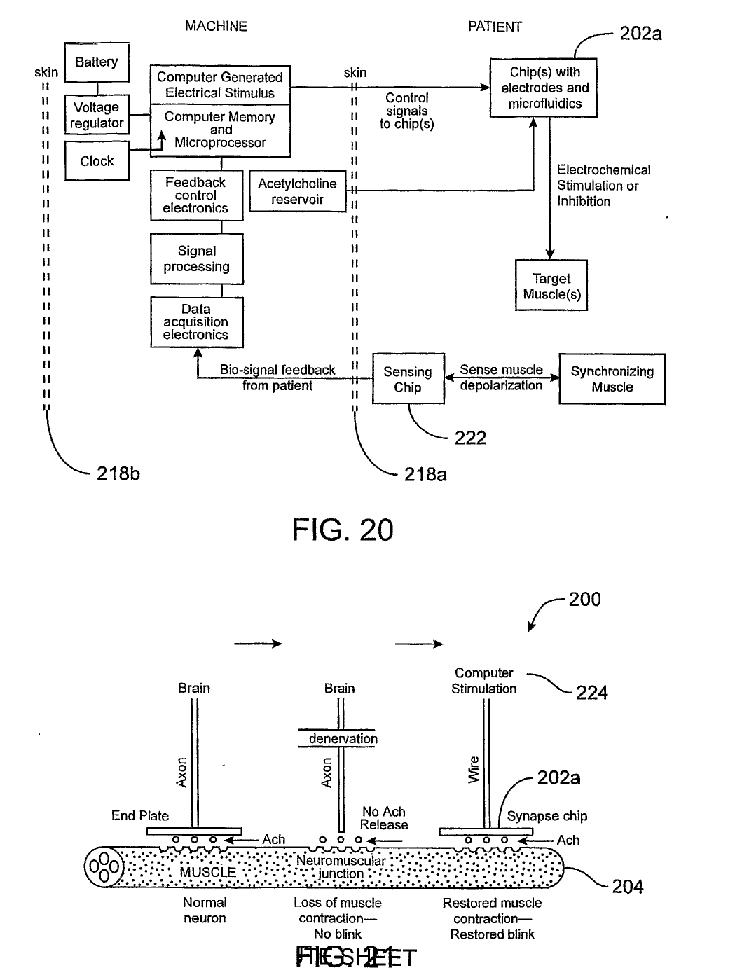

FIG. 20 presents still further details regarding selected components of an exemplary embodiment of system 200. System 200 can utilize micro-electro-mechanical system (MEMS) technology to create an indwelling microstimulator device, with the exemplary embodiments comprising a hybrid, indwelling microstimulator device or synapse chip structure 202(a) to deliver combined electrical and chemical stimulation when placed on or in engagement with a denervated muscle. A sensing chip 222 may be placed on or in engagement with a nearby functional muscle to signal the artificial synapse chip to induce a synchronous response in the affected muscle. Some or all of the implanted chips and components may be integrated into a wireless subdermal system. Simple embodiments may limit the complexity of the implanted components by transmission of electrical energy and or chemical agents through skin at a skin interface location 218a, while other embodiments may employ more implanted components as indicated schematically by skin interface location 218b.

As schematically indicated in FIG. 21, system 200 may be used to ameliorate the denervation of a muscle 204. When the bodies neurons are functioning, muscle contraction is initiated by signals from the brain. In a denervated muscle, contraction (and the associated movement, such as a blink) is no longer provided. By providing system 200, hybrid chemical and electromagnetic muscle contraction can be controllably effected in response to signals from a controller 224 of the system, with the chip structure 202a adjacent the muscle 204 acting as an artificial synapse.

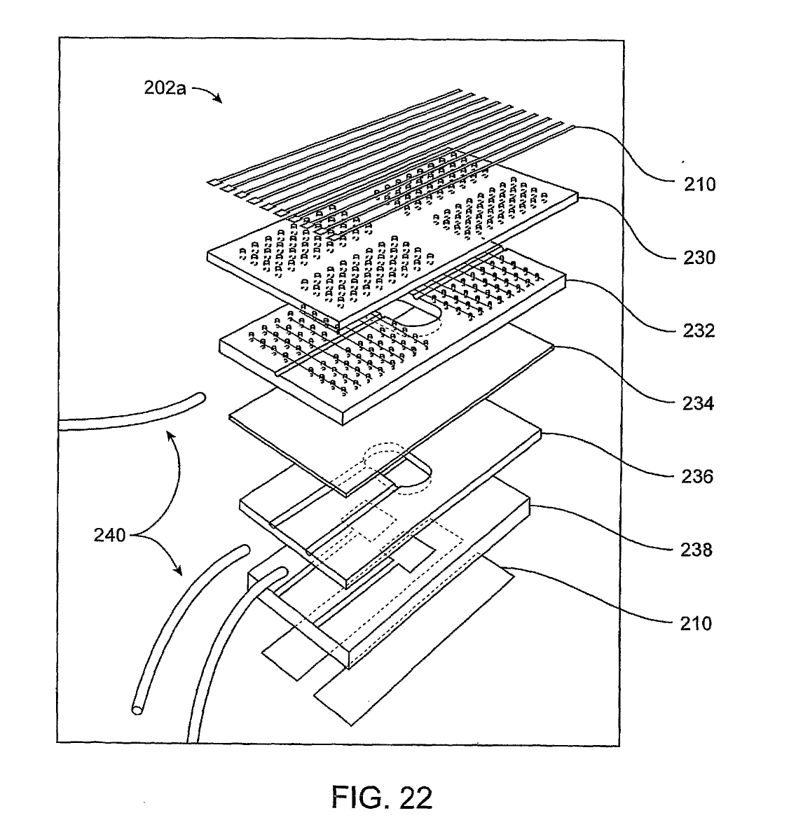

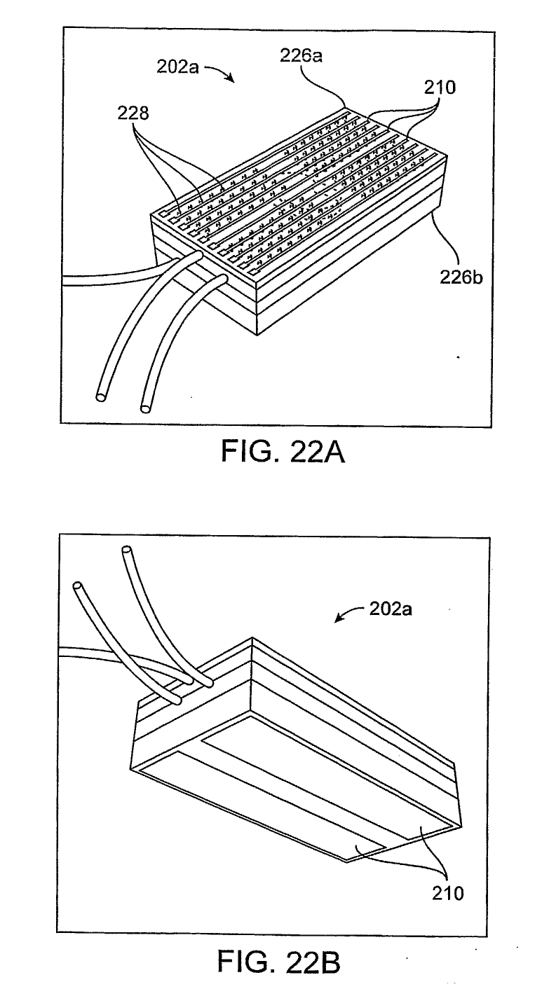

An exemplary synapse chip structure 202a for implantation adjacent an orbicularis oculi is illustrated in FIGS. 22, 22A, and 22B. In general, muscle implant chip structure 202a comprises a thin body having opposed major surfaces 226a, 226b with electrodes 210 thereon, so as to apply electrical stimulation from the anterior and posterior surface of chip structure 202a when implanted over the pretarsal orbicularis of both the upper and lower eyelids. An array of orifices 228 are in at least one of the major surfaces 226a. Exemplary chip structures 202a may have an overall length of at least 10 mm or more, for example having dimensions of 15 mm long×5 mm wide×0.5 mm depth. The components of chip structure 202a seen in the exploded view of FIG. 22 include electrodes 210, a glass top 230, a chemical reservoir and orifice layer 232, a deflectable membrane 234, a layer having an opening defining a water (or other working fluid) electrolysis/hydrolysis chamber 236, and a layer supporting electrolysis/hydrolysis electrodes 238. Chemical agent and working fluid supply flow is provided through lumens of fluid lines 240. As generally described above known microfluidic device and MEMS fabrication techniques can be employed to produce the channel network and reservoir of chip structure 202a.

MEMS technology and devices allow precise delivery of reproducible small volumes of bioactive substances. Such MEMS implants are capable of delivering from as little as zeptomole (10⁻²¹ mole) quantities, which can be equivalent to single vesicle quantities of bioactive substance. Much larger quantities can also be delivered, with many embodiments delivering nanoliter or microliter quantities of chemical agent fluids.

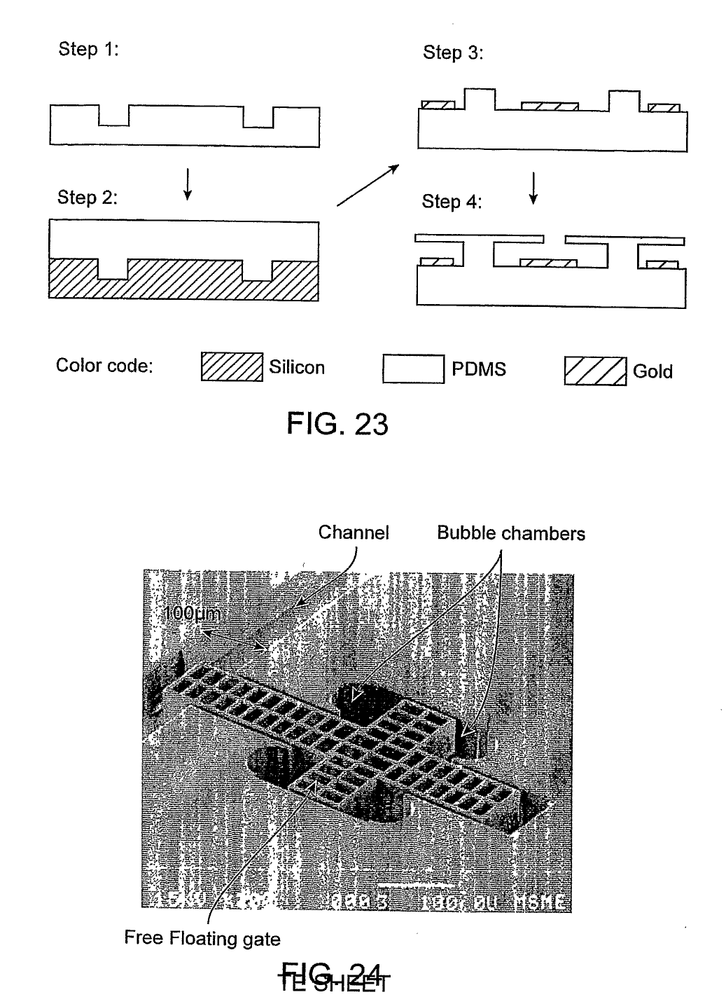

Referring now to FIG. 23, existing MEMS technology allows the microfabrication of a hybrid biocompatible chemical and electromagnetic implant chip structure 202a for use as an artificial synapse chip. This hybrid chemical and electromagnetic stimulation chip will be able to deliver adequate amounts of Ach (or other agents) to the denervated orbicularis (of a human or animal, such as a rabbit model) and create the appropriate microenvironment to initiate the cascade of events that results in muscle contraction with limited or no pain, spasticity, or persistent eyelid closure. Such chips may be fabricated with a size and shape of the implant described above, with appropriate modifications for the size of the patient anatomy, intended use, and the like. Chemical stimulation may be controllable variable to determine what concentration and volume of Ach should be delivered for a particular implant to create a blink (or other desired movement or effect). Appropriate concentrations and quantities of chemical agents may be affected by the size and distribution of the orifices. Fortunately, MEMS processing is very versatile and allows development of a wide range of these factors.

Hybrid stimulation chips may provide an electrical stimulation (optionally from an external computer board, an implanted microprocessor and battery source, or the like) and microfluidic delivery of acetylcholine via microapertures (the fluid movement optionally powered and controlled from the same or a different external computer board, from the same or a different implanted microprocessor and battery source, or the like). A variety of materials may be used for these biomedical devices. While silicon is convenient for testing and prototyping, its rigidity and brittle nature may not be ideal for implantation. Gold eyelid implants work well when they are slightly curved, a geometry which is not easily fabricated using silicon planar fabrication methods. Device weight is also a consideration for comfort. For these reasons, a plastic may be a better material, such as polydimethylsiloxane (PDMS), because of its material properties and biocompatibility. Micro-molded devices including embedded electronic subsystems may be particularly beneficial.

FIG. 23 illustrates some of the optional steps that may be used in the MEMS manufacturing approach, shown in cross-section. In step 1, construction may start using MEMS techniques of thin film deposition, lithographic patterning, and etch to create silicon mold-masters with features such as reservoirs, microfluidic channels, and microorifices. The use of lithographic patterning allows construction of feature sizes in the mold ranging from 2 microns to over 2 millimeters. In step 2, Polydimethylsiloxane (PDMS), a biocompatible polymer that can be easily spun into thin layers, can be poured into the silicon mold masters and subsequently polymerized to produce a robust but flexible film. This film can then be peeled from the silicon mold and further processed. Step 3 illustrates gold metal traces, for both electrical stimulation and to initiate fluid ejection through the microapertures, that can be deposited onto the free-standing PDMS using a technique known as shadow-masking. As seen in step 4, Multiple layers of PDMS can be assembled to produce channels, cavities, and other microfluidic features.

Referring now to FIG. 24, the implanted microfluidic network may include any of a wide variety of components, including a variety of different valves, pumps, and the like. The electrolysis/hydrolysis bubble actuated, free-floating gate valve illustrated here can be driven open or closed along an axis by energizing electrolysis/hydrolysis electrodes within selected bubble chambers. Such electrolysis/hydrolysis can employ low voltages, and by actuating these devices with small bubble sizes, the electrical power use can be within the capabilities of an implanted battery. For example, the power for operating a microvalve using electrolysis/hydrolysis can be less than 500 μJ and 5 volts. This indicates that such a valve might operate continuously for a year using a watch battery. The efficiency of using electrolysis/hydrolysis is also beneficial, with power and current on the order of 200 μW and 40 μA to form bubbles at 5 V. A wide range of alternative microvalve structures might be used, including a solenoid valve capable of high actuation speed such as those available commercially from Lee Company, CT. A relatively simple microfluidic channel network could, for example, drive chemical agents toward the fluid transmission surface using a pressurized reservoir or other chamber, with the chamber optionally being implanted behind the ear.

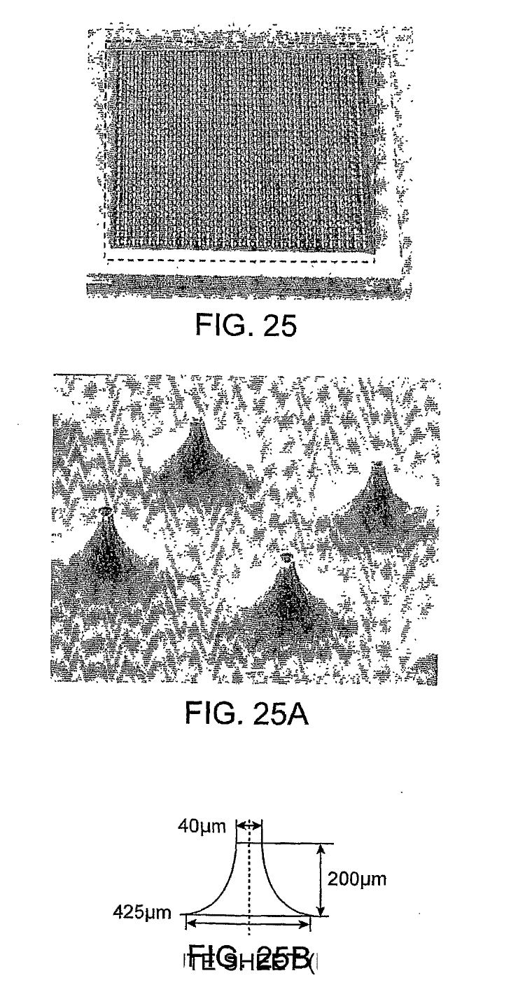

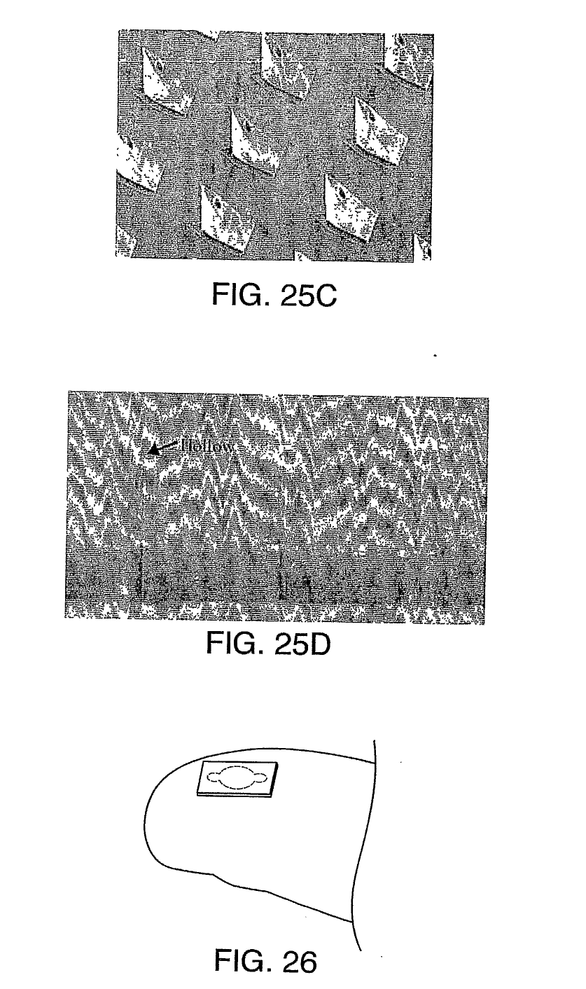

Referring now to FIGS. 25, 25A, and 25B, a simple design of microneedle arrays may be used to deliver chemical agents through the fluid transmission surface of the synapse chip. The arrays may include 400 needles/cm2 that would deliver the Ach uniformly across the pretarsal orbicularis. The presence of the needles, which are 200 μm long with a lumen diameter of 40 μm, may help ensure that the muscle tissue will be contacted diffusely and immediately by the Ach as it is injected, thereby helping to provide a rapid response. Alternatively, an array of simple orifices may be sufficient and easier to fabricate than the microneedles. Alternative fluid transmission surface structures (and related fluid channel system components) may also be used. For example, an alternative array of silicon microneedles from Silex Microsystems of Sweden is shown in FIG. 25C. Alternative microneedle materials may also be used, including PDMS or other polymers, as can be more fully understood with reference to an article by Kuo and Chou entitled “A Novel Polymer Microneedle Arrays and PDMS Micromolding Technique,” Tamkang Journal of Science and Engineering, Vol. 7, No. 2, pp. 95-98 (2004). Still further alternative existing structures may also be employed in the hybrid systems described herein. For example, a Chronojet™ drug delivery device from Debiotech of Switzerland is illustrated in FIG. 26. This supplier may be developing insulin and other drug delivery devices, components of which may be used (and/or modified for use) in the systems described herein.

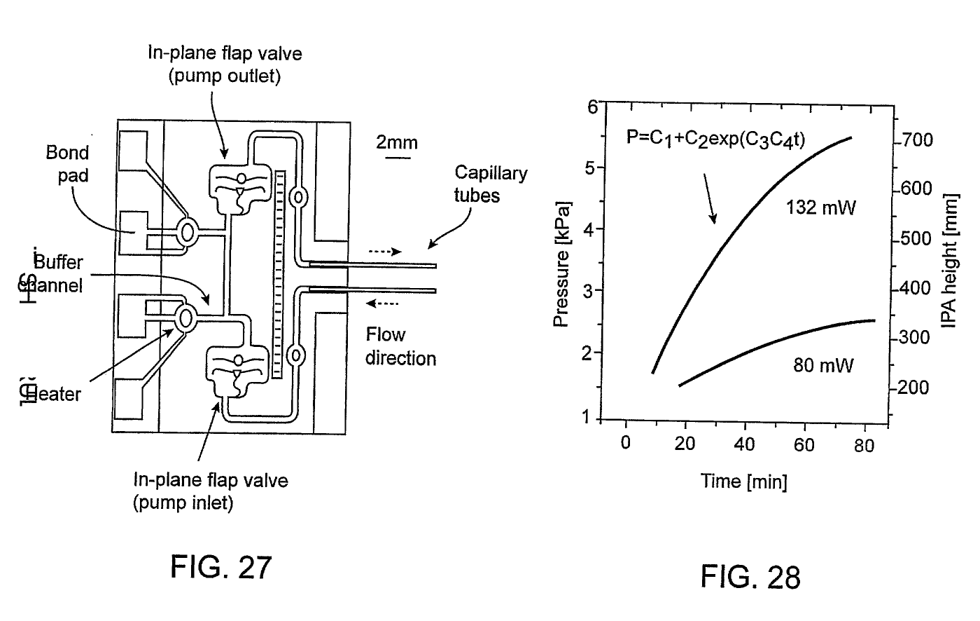

Referring now to FIGS. 22 and 27-30F, a wide range of micropump structures may be included in the synapse chip. Optional pumps may make use of electro-osmosis, electrophoresis, electrolysis bubbles, positive displacement structures such as pistons or diaphragms, and/or a pressurized chamber. One attractive approach is a displacement pump that effects movement by generation of gaseous H2 and O2 from water by electrolysis/hydrolysis, similar to the gate valve actuation of FIG. 24. In the embodiment of FIG. 22, the electrolysis/hydrolysis-induced growth of a gas bubble oscillates membrane 324, causing direct fluid displacement in an array of reservoirs separated from the electrolysis/hydrolysis chamber(s) by the membrane. The membrane movement in the reservoirs directly displaces Ach (or another chemical agent) and ejects it from the microorifices or microneedles of the synapse chip. In the alternative embodiment of FIG. 27, growth of a stable electrolysis/hydrolysis bubble in combination with two check-valves provides a positive displacement pump. Performance of an exemplary embodiment is graphically shown in FIG. 28. An advantage of electrolysis/hydrolysis is that it requires very little power to generate a relatively large displacement.

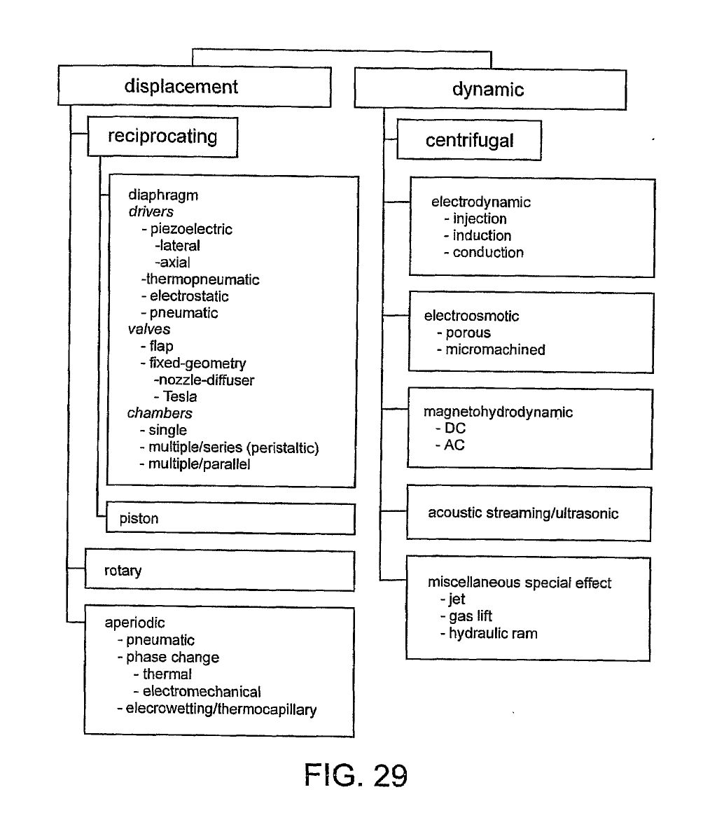

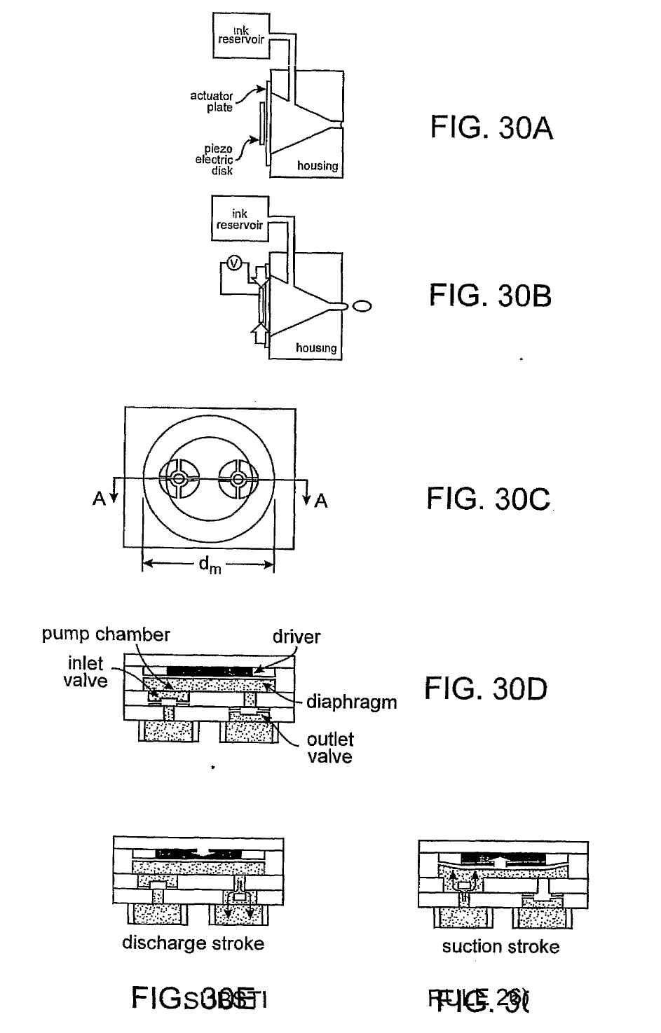

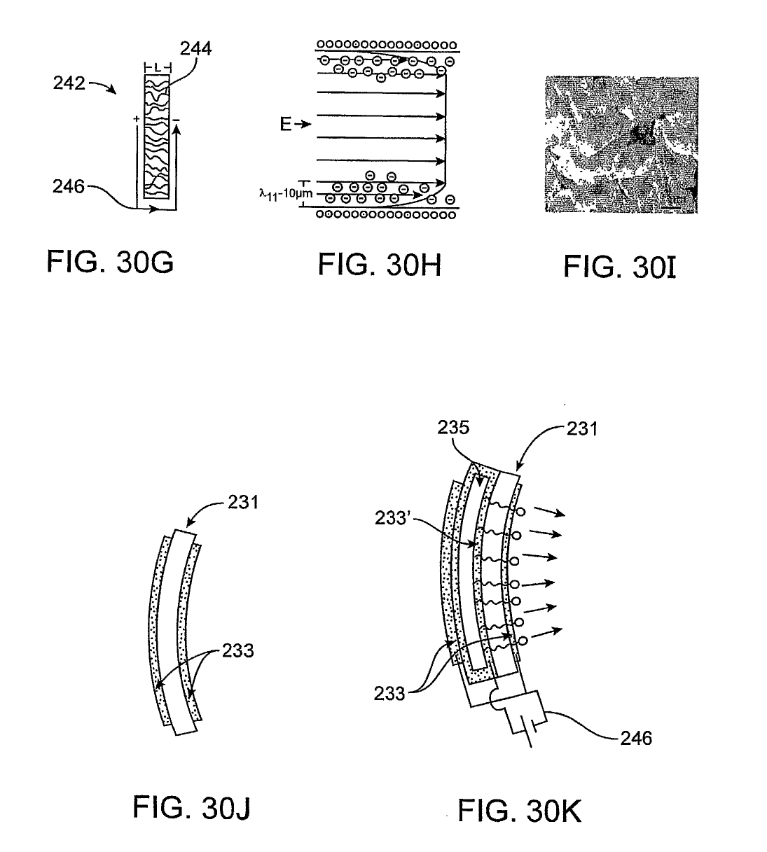

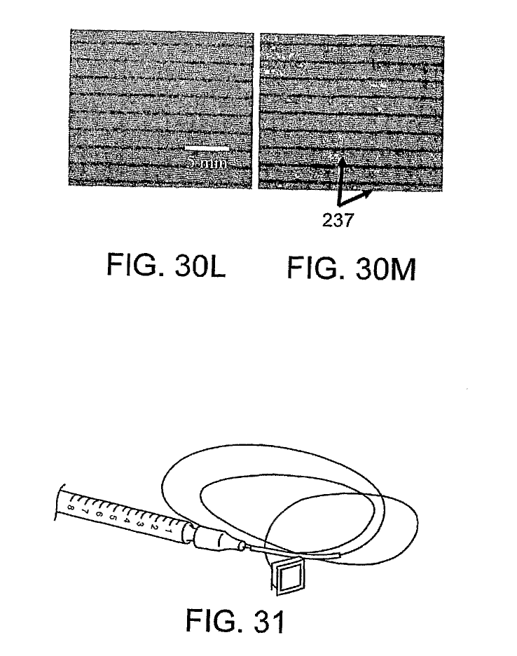

Still further alternative pumps based have also been developed which may be suitable for use in the systems described herein. As can be more fully understood with reference to an article by D. J. Laser and J. G. Santiago entitled “A Review of Micropumps,” J. Micromech. Microeng. 14 (2004), R35-R64, a variety of pump types, sizes, and performance characteristics may be selected. FIG. 29 provides classifications of candidate micropump types. FIGS. 30A and 30B schematically illustrate a displacement pump similar to that used in an ink jet printhead, in which the volume of the reservoir or chamber is varied using a piezoelectric disk actuator to deform a plate that seals the back side of the chamber. Surface tension at the ejector orifice (on the right side) acts as a check valve to rectify the flow, as may be more fully understood from U.S. Pat. No. 4,266,232. A reciprocating displacement micropump is shown in FIGS. 30C and 30D in top and side section views, respectively. FIGS. 30E and 30F show this pump in discharge and suction strokes. During the discharge stroke, the driver acts to reduce the pump chamber volume, expelling working fluid through the outlet valve. During the suction stroke, the pump chamber is expanded, drawing working fluid in through the inlet valve. Other techniques for delivering fluids include electroosmotic pumps. As can also be for fully understood from the Laser article (and/or as can be understood in more detail from the references cited therein), some synapse chips may use electromagnetic stimulation in combination with microfluidics.

Another potential pump option which may be used to direct fluid through the fluid transmission surface are integrated planar electroosmotic (EO) pumps, as can be understood with reference to FIGS. 30G-30M. Electroosmotic pumps are compact, can have no moving parts, and can be integrated into a wide range of structural and packaging designs. EO pumps can also be designed and operated using a variety of pumping substrates. Exemplary EO pumps may be fabricated using glass-particle-packed fused silica capillaries, porous borosilicate glass, in situ polymerized porous monoliths, and/or planar or porous silicon. Such pumps may, for example, have advantageously low power per flow rate, particularly when pumping with integrated EO pumps related to those that can be used in polyelectrolyte membrane fuel cells. The latter pumps can use less than 10% of the fuel cell power to completely clear fuel cell cathode channels of liquid water even at high current density. Detailed models of the flow rate, current, and pressure of electroosmotic pumps in porous materials may be available, allowing the performance of these pumps to be compared to dozens of other micro- and miniature pumps. EO pumps can, for example, move aqueous solutions ranging from less than 1 uM to 0.5 M solutions, and can pump both pure and aqueous solutions of organic solvents (e.g., acetone and methanol).

An exemplary EO pump structure and its operation are illustrated schematically in FIGS. 30G and H, respectively. EO pump 242 can, for example, comprise a porous pumping substrate 244 to which an electrical field 246 is imposed using two electrodes on either side of the porous substrate. The flow through the pump can be modeled as many cylindrical microchannels in parallel, with the flow shown in FIG. 30H schematically illustrating flow in one pore of the porous substrate. FIG. 30I shows a porous structure having pores on the order of 1 micron, such as may be included in an exemplary EO pump having a porous glass substrate. EO pumps can use ion drag in micro- and nano-scale flow channels to pump electrolytes. EO flow is the motion of an electrolyte caused by the interaction of an external electric field with the diffuse charges of electrical double layers (EDLs) which form at electrolyte/surface interfaces. The EDL's characteristic thickness is the Debye length λ_{D}.

A flexible porous substrate in an integrated EO pump as shown in FIGS. 30J (showing a pump structure including sputtered porous platinum electrodes for electroosmotic pumping action) and 30K (showing the assembled device including the drug reservoir, external electrodes for tissue stimulation, and internal electrode/pump anode). In FIG. 30J, a flexible porous polymer frit 231 is between two sputtered platinum electrodes 233. In FIG. 30K, a liquid reservoir 235 and additional electrode is added, with the two external electrodes 233 being electrically coupled together or shorted. In this configuration the electroosmotic pump is directly adjacent to a reservoir which stores the drug in aqueous solution. The pump structure is integrated with (flexible) electrodes that provide both the ionic current for electroosmotic pumping as well as the electrical potential stimulation of nearby tissue. Two outer porous metal sheaths form the outer muscle stimulation electrode. These are also the pump cathode. The inner porous metal electrode is shielded from the tissue and serves as the pump anode.

The design of FIGS. 30J and 30K may offer advantages. First, the potential of the outer electrode/pump cathode (in contact with the surrounding tissue) can be varied independently of the pump anode. This may allow for independent control of electrical stimulation signals and pump actuation voltage. For example, a 2 V potential can be applied to the external (pump cathode) electrode, while a 10 V potential is applied to the internal pump electrode (the pump anode). The surrounding tissue experiences a 2 V potential (relative to ground), while the pump reacts to a 10 V potential differences. Real-time control of the electrodes may be applied to provide, for example, 100 ms pump electrode pulses and effect delivery of 10 to 1000 nl aliquots. The outer electrode might be pulsed relative to ground (and independent of the pump anode) to achieve 100 to 500 ms duration electrical stimulation of surrounding tissues. These pulses could be separated by about 1 to 2 sec intervals, or initiated in response to sensing of a contralateral blink or the like. Pulse shape and the phase lag of both pump and stimulation pulses can be easily controlled as well as pulse shape and integrated current. Between pulses, small (e.g., 2 to 4 V) DC values of pump potentials may optionally be applied in order to prevent back diffusion of molecules into the reservoir and mitigate biofouling of the pump membrane. Alternatively, short duration (<10 ms) pulses of large pump potentials (e.g., 20 V) (not detectable/affecting external tissue) might be applied in order to clear or inhibit biofilms.

Referring now to FIGS. 30L and 30M illustrate the use of the implant structure of FIGS. 30J and 30K for controllably releasing fluids. These figures show a fluid transmission surface of a machined porous glass pump substrate. The horizontal grooves shown on the surface may not be present in many embodiments of the hybrid chemical and electromagnetic muscle stimulation synapse chip. A 5 V applied potential drives an aqueous solution from a reservoir behind the pump, through the pump substrate, and out onto the top surface. More specifically, both faces of the substrate are covered with a sputtered layer of porous platinum that supplies good in-plane conductance while allowing liquid flow. As shown in FIG. 30M, upon application of 5 V, the substrates pumps aqueous solution from a reservoir behind the pump to the top face (resulting in visible water droplets 237).

An analysis of power for an electroosmotic pump design, including pump pressure and flow rate requirements, temporal response, flow-rate-per-power and thermodynamic efficiency can also be performed. There may be a trade off between pump area and power efficiency. For example, about 10 nl doses every second may be achieved with 100 ms response (duration of dosage pulse). A pump with an area of less than one millimeter squared might achieve this performance at pH=7 and a 1 mM concentration of background aqueous electrolyte with a 30 V applied internal pump potential (again, the tissue does not experience this potential). The peak generated pump pressure in this 100 ms pulse may be on the order of 100 kPa. The thermodynamic efficiency of such pumping may only be about 1%, but the power requirement can still be quite reasonable. For example, such a 10 nl pulse may use only about 100 micro Joules of energy, so that a AAA Nickel-Cadmium battery could achieve over 10 million pulses (optionally providing a life of 150 days at 1 Hz).

The dependence of energy-per-pulse and operation power may scale as the applied voltage squared. For a given flow rate, pump voltage can be kept low by increasing pump area. For example, a 1 square centimeter pump can achieve a few microliters per second with 2 mW of power (650,000 pulses or one week with a AAA battery) at an applied pump/internal potential of 7 V.

While many of the above exemplary embodiments may employ sophisticated hybrid electrical and chemical MEMS structures to electro-chemically stimulate muscle tissue, alternative embodiments may make use of separate electrical and chemical components, and/or relatively simple devices. A simple chemical delivery implant employing a syringe to deliver a chemical agent is illustrated in FIG. 31. Dosing may be facilitated by a commercially available micropipette, such as the Magic Assist Pipette™ continuously adjustable digital microliter pipette from Rainin, Inc., of Oakland, Calif. Such a device may allow dosing of from 0.1 μL to 200 μL.

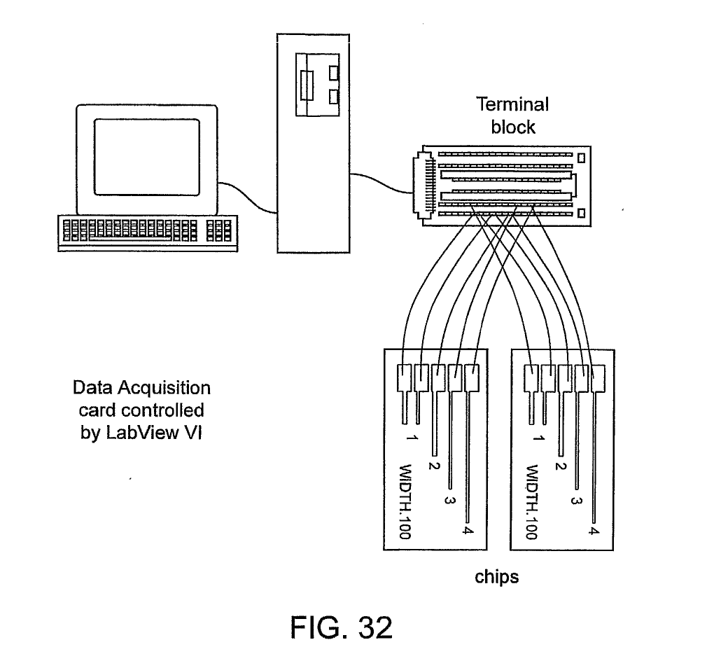

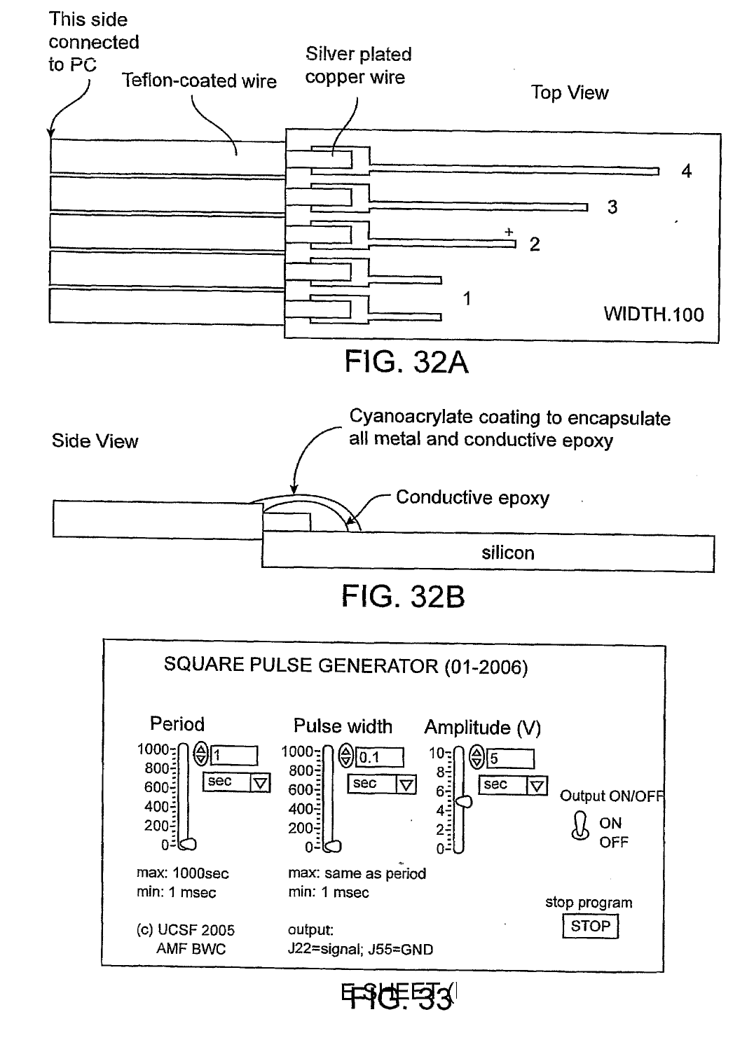

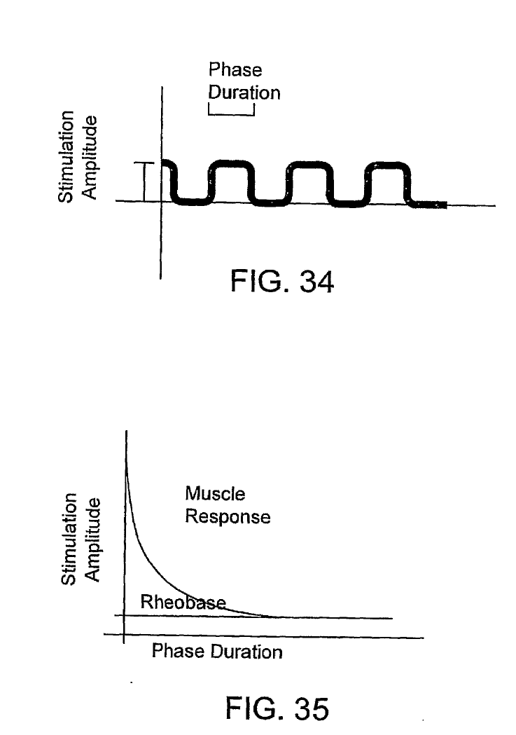

FIG. 32 illustrates external, general purpose computer-controlled electrical stimulation system components, which may be separate from the chemical release system. Some or all of these electrical components may alternatively be included in a hybrid system. In general, the electrical stimulation or MEMS devices can be operated using a programmable National Instruments computer board controlled by custom-written LabView software. The use of an external, programmable control may allow varying stimulation voltages and currents, as well as adjustments to the actuation of the Ach dispensing through the micro-orifices. FIG. 32A and 32B show an electrical stimulation implant, and FIG. 33 illustrates a user interface for setting electrical stimulation signal properties. While the electrical stimulation signal of FIG. 34 is a simple direct current square wave cycle, a variety of alternative potentials and signal waveforms might also be implemented. Exemplary electrical stimulation parameters for experiments or use may have amplitudes in a range from about 0 to about 10 volts, a phase duration in a range from about 1 microsec to 1000 sec, and a stimulation frequency period in a range from about 1 microsec to about 1000 second. An exemplary strength-duration curve for a skeletal muscle is shown in FIG. 35.

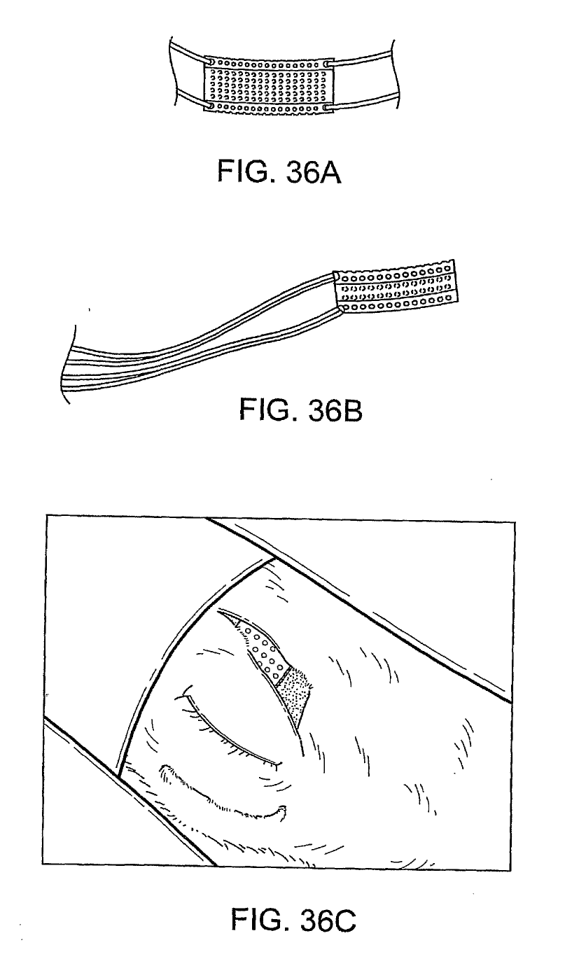

Referring now to FIGS. 36A and 36B, alternative electrical stimulation implants are shown. These exemplary structures comprise stainless steel plated with gold, along with a polyimide tape array. These rabbit-model implants have major surface dimensions of 20 mm×5 mm, and 15 mm×5 mm, respectively. FIG. 36C illustrates implantation of such a structure in an eyelid of a rabbit.

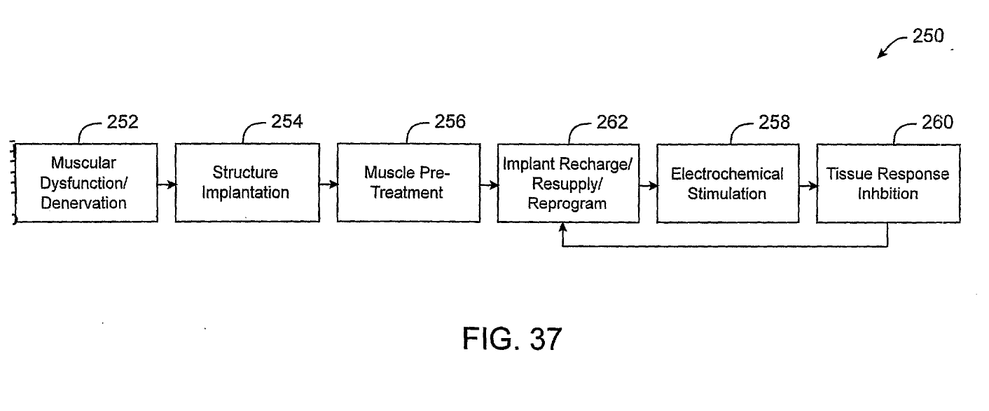

Referring now to FIG. 37, an exemplary method 250 for treatment of the present invention is initiated in response to a muscular denervation or other dysfunction 252. Any one or more of the stimulation device structures described herein is implanted 254. Optionally, the implanted device may be used to pre-treat the muscle 256, such as to inhibit or counteract atrophy. The implanted structure(s), autonomously or, at least in part under the control of external components of the system, chemically and/or electrically stimulate the muscle 258. Encapsulation or other deleterious tissue responses may be inhibited 260. Operation of the system may be altered or extended 262 by, for example recharging an implanted battery using a through-skin wireless charger or the like, resupplying one or more chemical agents to (and/or removing one or more waste products from) associated implanted reservoir(s) using a syringe needle passing through the skin and a sealing reservoir membrane or the like, reprogramming an implanted processor wirelessly through the skin or the like, and/or other appropriate actions. Feedback may be provided from implanted components using wires or wireless telemetry.

### EXPERIMENTAL

### Experiment 1: Palsy Patients

4 subjects with denervated orbicularis oculi were tested with electrical-only stimulation at predetermined locations in the preseptal and pretarsal orbicularis oculi, identified by anatomic landmarks. A typical data set is shown below, in this case for a patient denervated on the right side as shown in Table I.

TABLE I

| Position of Electric | | | Right Eyelid | |
|---|---|---|---|---|
| Stimulation | Phase Duration | Current | Movement | Pain |
| 5 mm superior to the upper lid | 0.05 msec | Up to 11.8 mA | No movement | 6/10 |
| punctum | 0.01 msec | Up to 15 mA | No movement | 6/10 |
| 5 mm superior the upper lid | 0.01 msec | Up to 26.7 mA | No movement | 6/10 |
| margin at mid pupil | | | | |
| 10 mm lateral to the lateral | 0.01 msec | Up to 20.4 mA | 1 mm twitch | 5-6/10 |
| margin | | | | |
| 10 mm inferior to lower lid | 0.01 msec | Up to 26.7 mA | No movement | 7/10 |
| margin at mid pupil | | | | |
| Preseptal Surface | 0.01 msec | Up to 36.9 mA | No movement | 7/10 |
| Electrode, Upper lid | | | | |

The levels of stimulation in the table were the limits of stimulation tolerable to the patient. Complete functional blinks were not elicited. Notably, a fill body startle type movement was elicited at the upper limits of electrical intensity at all test positions above. The results in the other three patients were similar. No complete eyelid closure blink was elicited using stimulation parameters that could be tolerated.

The amount of electric stimulation required to produce a functional complete closure blink of the denervated orbicularis oculi does not appear tolerable in humans, indicating the insufficiency of electric stimulation alone for the production of a functional blink.

### Experiment 2: Electrical Stimulation In the Denervated Rabbit Model

To determine if an implantable prototype device capable of delivering electrical stimulation could elicit a complete closure blink of a denervated orbicularis oculi muscle in New Zealand White Rabbits, a rabbit model was used. The rabbit model was selected because of the similarity of the structure and function of their eyelids; specifically the distribution of neuromuscular junctions and muscle fiber type of the orbicularis oculi when compared to humans.

### Methods

### Facial Nerve Denervation

a) Two white New Zealand female rabbits were anesthetized by using 3-5% isofluorane inhalation and ketamine/xylazine and monitored by Heska monitor (SP02, heart rate, and rectal temperature). b) A pre-auricular incision was made the facial nerve was surgically sectioned and a five millimeter section was eliminated. The upper eyelid opens when the innervation to the orbicularis oculi is severed creating 6 millimeters of lagophthalmos.

### Electrical Stimulation Experiments

Methods: a) A micro-fabricated electrical stimulation unit with a main body of silicon measuring 6 mm in length, 3 mm in height and 1 mm thick was placed in the upper and lower eyelids of a rabbit (see FIGS. 32A, 32B). The silicon chip surface had electrodes made of gold with line widths greater than 50 microns. b) a program created utilizing Lab VIEW (National Instruments) was run on a PC (see FIG. 36) and delivered square wave direct stimulation with an adjustable pulse width from 1 millisecond to 1000 sec and a voltage range of 1 millivolt to 10 volts. A standard volt meter was used to confirm that the system was functioning.

### Experiment 2a

Two weeks post-denervation, one prototype chip with the electrical stimulation delivery facing upwards was placed in the upper and lower lid, with externalized wires to enable stimulation to be controlled by a computer board.

Results: Stimulation produced a localized muscle contraction of the orbicularis oculi, evidenced by a twitch of the upper and lower eyelids.

Discussion: Since the pretarsal fibers of the orbicularis oculi only span a third of the length of the muscle, and local electric stimulation can only travel the length of individual fibers, stimulation across a greater portion of the entire length of the muscle may elicit effective contraction. Other possible reasons for limited response to stimulation may include an insufficient size and layout of the gold electrodes, any defect in the connections between the stimulation unit and the chip electrodes, and any localized loss of insulation of the wires causing the wires to short circuit prior to current reaching the chip electrodes.

### Experiment 2b

Four weeks post-denervation, three chips were placed in the right upper lid, and 10 Volts delivered to each chip. Both stimulation chips in the up and down positions were tested on the same day and then two days after placement. Video documentation was performed.

Results: A slight twitch of the right upper eyelid was seen on the day of chip placement during electric stimulation. Two days later the same stimulus produced a more robust twitch, but not a full effective blink.

### Experiment 2c

A second prototype was fabricated from stainless steel sheet electroplated with gold, and was 20 mm long by 6 mm wide (see FIG. 36A). It was constructed to provide electrical stimulation on both top and bottom surfaces and voltage was delivered using the computer board stimulation system. Video documentation was again performed.

Results: Using this device to deliver electric stimulation at 5 volts with a phase duration of 70 msec resulted in a complete, natural appearing blink that was reproducible.

### Experiment 2d

A third prototype was fabricated from stainless steel sheet electroplated with gold that was 10 mm long, 6 mm wide and was conductive on both top and bottom surfaces.

Results: Using this device and the computer board stimulation system, no combination of current voltage or phase duration could create a complete blink. Of note, the heart rate went from 180 bpm to 220 bpm during stimulation testing.

Discussion: The above experiments indicate that the amount of electrical-only stimulation to create eyelid movement is painful. To enhance the effectiveness of the electrical component of the device, it benefits from coverage over a significant portion of a length of, preferably the majority of, or even as much of the orbicularis as possible (the orbicularis having a length of about 20 mm in humans; 15-20 mm in rabbits) and should deliver impulses to both the anterior and posterior surface of the device. Adding the chemical component may alter these requirements.

### Experiment 3: Ach Stimulation In the Denervated Rabbit Model

To determine the response of denervated orbicularis oculi in rabbits to stimulation with varying dosages of Miochol-E (acetylcholine chloride, injectable, Novartis pharmaceuticals) the FDA approved Miochol-E in 10 mg/ml, or 0.055M Ach (molar mass of 182 g/mol) was used to test the effects in the denervated rabbit model.

Methods: The rabbit was sedated in standard fashion, an eyelid incision was made 5 mm from the lash line and the Miochol E was injected into the orbicularis oculi pocket. Retesting was done at six weeks.

Results are shown in Table II.

TABLE II

| | Vol Ach Delivered | | | | | | | |
|---|---|---|---|---|---|---|---|---|
| Dilution | | | | 10 | 25 | 50 | | |
| of Ach | 0.5 μl | 1 μl | 5 μl | μl | μl | μl | 100 μl | 200 μl |
| Full Strength | N | N | N | N | N | N | N | C × 1 |
| Ach (10 mg/ml) | | | | | | | | minute |
| 1:1 | N | N | N | N | N | N | N | |
| 1:2 | N | N | N | N | N | N | N | |
| 1:5 | N | N | N | N | N | N | N | |
| 1:10 | N | N | N | N | N | N | N | |

N = no response;
C = complete eyelid closure

Discussion: The electrochemical delivery device should deliver microliter amounts that are concentrated enough to result in a complete blink, but not so great that they overwhelm the natural inactivation of the delivered acetylcholine by acetylcholinesterase.

### Experiment 4: Synergistic Action of Electric Stimulation And Ach

A possible synergistic interaction between Ach and electric stimulation were observed as follows. After having injected an amount of Ach into the right denervated orbicularis oculi that produced no reaction, electric stimulation was delivered to the muscle at an intensity previously unable to produce a definitive blink. The eyelid progressively closed over the course of three stimulated partial blink motions until it closed tightly for four minutes prior to relaxing to pre-testing height.

In a subsequent test session, the right denervated orbicularis was first tested using electrical stimulation alone (prior to any Ach testing) as shown in Table III.

TABLE III

| | Phase Duration | | | | | | | |
|---|---|---|---|---|---|---|---|---|
| | | 1 | 10 | 20 | 30 | 50 | 70 | 100 |
| Amplitude | 0.5 msec | msec | msec | msec | msec | msec | msec | msec |
| 1 volt | T | T | T | T | T | T | T | T |
| 2 volts | T | T | P | T | T | T | T | T |
| 3 volts | T | P | P | P | P | P | P | P |
| 4 volts | T | P | P | P | P | P | P | P |
| 5 volts | T | P | P | P | P | P | P | P |
| 6 volts | T | P | P | P | P | P | P | P |
| 7 volts | T | P | P | P | P | P | P | P |
| 8 volts | T | P | P | P | P | P | P | P |
| 9 volts | T | P | P | P | P | P | P | P |
| 10 volts | T | P | P | P | P | P | C | P |

T: Twitch;
P: Partial Eyelid Closure;
C: Complete Eyelid Closure

Ach testing was then carried out, and the results are shown in the table in Table II above. After the delivery of 200 ul of Ach to the right denervated orbicularis oculi, the right orbicularis oculi was allowed to relax to baseline position prior to Ach stimulation. No saline flush was performed. Electric stimulation was given at 10 msec phase duration at 3 volts. Reproducible complete closure blinks were induced.

### Experiment 5: Chemical Stability of Ach At Body Temperature

To determine the stability of acetylcholine at 37 C to help guide the reservoir sizes and locations, aliquots of reference acetylcholine and standard strength Miochol-E (acetylcholine, injectable, Novartis Pharmaceuticals) were stored in sterile glass containers at 37 C. Ach concentration was analyzed by HPLC with UV detection on days 0, 1, 3, and 6, and 14 days.

Results: Control Ach Peak Area: 58950; Miochol-E Peak Area after storage at 37 C for 14 days: 56494.

Discussion: Miochol-E appears to be stable for 14 days in sterile glass at 37 C.

### Conclusions From Experiments

The data indicates that the amount of electric stimulation alone required to produce a functional blink in both denervated humans and rabbits is painful. Ach delivered to the orbicularis in a diffuse fashion results in muscle contraction that create tonic, prolonged closure at certain concentrations and volumes. Combined electrochemical stimulation appears to provide benefits for producing an effective blink

All publications and patent applications cited in this specification are herein incorporated by reference as if each individual publication or patent application were specifically and individually indicated to be incorporated by reference.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended claims.

1. A hybrid chemical and electromagnetic device for electrochemically stimulating tissue, comprising:
(a) a structure having one or more orifices, said structure comprising at least one reservoir for holding at least one chemical agent, wherein said at least one chemical agent is ejected from said one or more orifices to said tissue, wherein said tissue is chemically stimulated by said at least one chemical agent; and
(b) an electromagnetic stimulation device for delivering an electromagnetic stimulus to said tissue, wherein said delivery of said electromagnetic stimulus is for electrically, magnetically or electromagnetically stimulating said tissue.

2. The hybrid device as set forth in claim 1, wherein said at least one chemical agent comprises a chemical transmitter, a neurotransmitter, acetylcholine, acetylcholineresterase, chorbechol, an element, calcium, or a combination thereof.

3. The hybrid device as set forth in claim 1, wherein said structure is implantable inside a body, wherein said tissue comprises a muscle tissue, wherein said at least one chemical agent is delivered directly to a motor end plate of said muscle tissue, and wherein said delivery of said at least one chemical agent bypasses a plurality of nerves and a neuromuscular junction of said muscle tissue.

4. The hybrid device as set forth in claim 1, wherein said tissue is at least partially from a cancer tissue, a facial muscle, an eyelid muscle, or an orbicularis oculi muscle.

5. The hybrid device as set forth in claim 1, further comprising a controlling device for providing control signals to said structure, wherein said control signals control said delivery of said at least one chemical agent and said electromagnetic stimulus to said tissue.

6. The hybrid device as set forth in claim 5, wherein said tissue is from a first muscle, wherein said hybrid device further comprises a sensor device for detecting changes in a second muscle, wherein said sensor device is communicatively connected to said controlling device, and wherein said control signals can be at least partially based on said changes detected by said sensor device.

7. The hybrid device as set forth in claim 6, wherein said changes in said second muscle comprises a change in voltage, a change in current, a change in movement, or any combination thereof.

8. The hybrid device as set forth in claim 6, wherein said control signals control delivery of said at least one chemical and said electromagnetic stimulus to said first muscle, and wherein said control signals synchronize said first muscle with said second muscle.

9. The hybrid device as set forth in claim 1, wherein said structure comprises a fluid transmission surface, wherein said fluid transmission surface is fluidically connected to said at least one reservoir.

10. The hybrid device as set forth in claim 9, wherein said fluid transmission surface comprises an array of protrusions, wherein said one or more orifices are located on said protrusions.

11. The hybrid device as set forth in claim 9, further comprising a drug eluting coating, wherein said drug eluting coating is on said fluid transmission surface.

12. The hybrid device as set forth in claim 1, wherein said at least one chemical agent changes a response of said tissue to said electromagnetic stimulus.

13. The hybrid device as set forth in claim 1, further comprising a pump configured to assist in delivery of said sat least one chemical agent.

14.-67. (canceled)

68. A method for electrochemically stimulating tissue, comprising:
(a) implanting a delivery device within a body, wherein said delivery device comprises one or more reservoirs and one or more orifices, wherein said one or more reservoirs are for storing at least one chemical agent to be released to said tissue, wherein said delivery device can be activated to release at least one chemical agent from said one or more reservoirs to said tissue through said one or more orifices;
(b) activating said delivery device to release said at least one chemical agent to said tissue for chemically stimulating said tissue; and
(c) delivering an electromagnetic stimulus to said tissue for electrically, magnetically, or electromagnetically stimulating said tissue.

69. The method as set forth in claim 68, wherein said activating said delivery device step occurs before or simultaneous with said delivering said electromagnetic stimulus step, wherein said released at least one chemical agent changes a response of said tissue to said electromagnetic stimulus.

70. The method as set forth in claim 69, wherein said tissue comprises muscle tissue, wherein said electromagnetic stimulus is for stimulating contraction of said muscle, wherein said released at least one chemical agent reduces the electric current or potential required for stimulating said contraction of said muscle.

71. The method as set forth in claim 68, wherein said releasing of said at least one chemical agent comprises releasing said at least one chemical agent in a plurality of stimulation cycles, wherein an amount of said at least one chemical agent released per each of said stimulation cycles is less than about 200 μL, from about 2 nL to about 100 μL, or from about 2 nL to about 10 μL.

72. The method as set forth in claim 68, wherein said tissue is from a first muscle, said method further comprising:
detecting said changes in a second muscle; and
activating said delivery device to release said at least one chemical agents to said tissue of said first muscle based on said detecting of said second muscle.

73. The method as set forth in claim 72, wherein said activating said delivery device chemically stimulates said tissue of said first muscle, wherein said first muscle is stimulated to synchronize with said second muscle.

74. The hybrid device as set forth in claim 68, wherein said at least one chemical agent comprises a chemical transmitter, a neurotransmitter, acetylcholine, chorbechol, acetylcholineresterase, an element, calcium, or any combination thereof.
